(19) United States
(12) Patent Application Publication
(10) Pub. No.: US 2004/0241636 A1
(43) Pub. Date: Dec. 2, 2004

(54) Monitoring gene silencing and annotating gene function in living cells

(76) Inventors: Stephen William Watson Michnick, Westmount (CA); Barbara Belisle, Moraga, CA (US); Marnie L. MacDonald, Pleasanton, CA (US); John K. Westwick, San Ramon, CA (US); Jane Elizabeth Lamerdin, Livermore, CA (US)

Correspondence Address: Isaac A. Angres, Suite 301, 2001 Jefferson Davis Highway, Arlington, VA, 22202, US

(21) Appl. No.: 10/856,620

(22) Filed: May 29, 2004

Related U.S. Application Data
(60) Provisional application No. 60/474,283, filed on May 30, 2003.

(51) Int. Cl. C12Q 1/68; C12Q 1/00; C12N 15/85

(52) U.S. Cl. 435/4; 435/455; 435/6

(57) ABSTRACT

The cell-based assays described in the present invention can be used to directly assess the sensitivity and specificity of the gene annotation reagent against its target, and to determine if a non-targeted gene participates in a pathway of interest or is functionally linked to another gene or protein. The combination of annotation reagents with such cell-based assays is useful for mapping genes (proteins) into cellular pathways on a genome-wide scale. Preferred assay embodiments include fluorescence or luminescence assays in intact (live or fixed) cells. Such fluorescence or luminescence assays include high-throughput or high-content assays for protein activity, subcellular localization, post-translational modifications, or interactions of proteins. Suitable assays may include protein-protein interaction assays; protein translocation assays; and post-translational modification assays. The invention can be used to assess the efficacy of any gene silencing experiment, to determine the level of gene silencing that is achieved, and to map novel genes into biochemical pathways, and to identify novel pharmaceutical targets. The results also demonstrate the feasibility of employing this strategy in genome-wide functional annotation efforts.

This application claims the priority benefit under 35 U.S.C. section 119 of U.S. Provisional Patent Application No. 60/474,283 entitled “Monitoring Gene Silencing And Annotating Gene Function In Living Cells”, filed May 30, 2003, which is in its entirety herein incorporated by reference.

### BACKGROUND OF THE INVENTION

This invention relates generally to the fields of biology, molecular biology, chemistry and biochemistry. Specifically, the invention is directed to methods for annotating gene function, mapping disease pathways, and validating pharmaceutical targets. In particular, the invention provides for the use of annotation technologies in combination With cell-based high-content or high-throughput assays as a method to map genes and proteins into biochemical pathways; to identify novel disease pathways; and to identify and validate novel pharmaceutical targets.

A wide range of biochemical tools, reagents, and technologies are now available for use in target validation. These tools enable selective or nonselective silencing, inactivation or activation of individual genes or proteins in living cells. The toolbox includes, but is not limited to, RNA interference (RNAi), small interfering RNAs (siRNAs), antisense probes, ribozymes, gene expression technologies, homologous and non-homologous recombination, natural and synthetic peptides and polypeptides, purified whole proteins, antibodies, intrabodies, suicide substrates, chemical modifiers and crosslinkers, and related approaches. For the purposes of this invention we will refer to such technologies and their associated reagents as “annotation technologies” and “annotation reagents”, respectively. Given a suitable form and delivery method, many of these reagents can be applied to biological systems—including living cells, tissues, or even whole organisms (McCaffrey A. P., et al., Nature Biotech 21: 639-643, 2003) in order to identify the biochemical, biological or phenotypic consequences of modulation of a particular gene or protein of interest.

Gene-silencing studies began in the mid-1980's with antisense technologies, wherein homologous RNA, DNA or chemically altered nucleic acids hybridize in a sequence-specific manner to targeted mRNA transcripts, thereby inhibiting their expression post-transcriptionally. In the early 1990's, nucleic acid molecules were used to directly target the transcriptional regulation of gene expression. ‘Triplex’ generating reagents opened the window for researchers to inhibit the transcription process itself by introducing a nucleic acid molecule that hybridizes to a specific sequence of DNA within a cell. RNAi methods are also based on nucleic acid technology, however unlike antisense and triplex approaches, the double-stranded RNA (dsRNA) activates a normal cellular process leading to a highly specific RNA degradation (Hannon, G. J., Nature 418: 244-251, 2002; Paddison, J. And Hannon, G. J. Cancer Cell 2: 17-23, 2002. Studies over the last several years have demonstrated that RNA interference is mediated by the generation of 21- to 23-nucleotide dsRNA molecules, termed small interfering RNA (siRNA) which can be directly introduced into cells or transfected as part of a suitable vector (Yu, J-Y, DeRuiter, S. L., & Turner, D. L., Proc. Natl. Acad. Sci. 99: 6047-6052, 2002; Paul, C. P., et al., Nature Biotech 29: 505-508, 2002). Because RNAi technologies can in principle allow the silencing of a gene given its sequence, RNAi has become an extremely popular research tool to annotate the function of novel genes on a large scale.

In addition to technologies that involve modulating the expression or stability of RNA, many methods and reagents exist for direct modulation of protein activity. These methods can also be used to modulate protein expression or activity in cells. Exogenous expression of wild-type (native) or mutationally-activated proteins has been used extensively to mimic the active state of a particular protein in living cells. This can be achieved by transfecting cells with plasmid-based expression vectors, or infecting with a retrovirus, adenovirus, or lentivirus encoding the protein of interest. Purified proteins may also be microinjected into cells (or organisms) to mimic the activated state of a protein and its cognate pathway.

Mutational inactivation of signaling proteins has also been described, and exogenous over-expression of these mutants has been widely used to block the activity of the endogenous homolog and its cognate pathway. This “dominant-negative” approach has been used extensively to probe the activity of several classes of proteins, including protein kinases (e.g. Kitamura, T., et al., Mol Cell Biol 18: 3708-3717, 1998; Wang, Q., et al., Mol Cell Biol. 19: 4008-4018), other nucleotide binding proteins such as GTPases, and transcription factors.

Other polypeptide-based approaches to protein activity modulation have been described. Introduction of peptides comprising structural or protein binding elements of transcription factors, kinases, or E3 ubiquitin ligases will block protein-protein interactions and activities downstream from these elements in signal transduction pathways. For example, elements of protein kinases known as aJ and HJ loops are highly conserved at the structural level, but divergent at the amino acid level between particular kinases. Introduction of isolated polypeptides with amino acid sequences matching these structural elements can block the cellular activity of the kinase, presumably through competitive interactions with substrate proteins. This has led to the application of these tools as inhibitors of specific kinases and the pathways in which they operate [e.g. U.S. Pat. Nos. 6,723,830, 6,723,694, 6,174,993].

Specifically targeting proteins for degradation is another analogous approach to siRNA targeting of messages. Engineered proteins which bridge a protein of interest to proteasomal degradation have been described (Sakamoto, K. M. Kim K B, Verma, R., Ransick, A. Stein, B., Crews, C. M., and Deshaies, R. J. Mol. Cell Proteomics, 12:1350-1358, 2003).

Importantly for the pharmaceutical industry, gene annotation technologies offer the hope of identifying and prioritizing novel targets for pharmaceutical development. However, to realize this goal on a large scale, there are three key needs that remain to be fully realized: (1) reliable and specific gene annotation technologies that can be applied on a large scale; (2) reliable and scalable methods for directly detecting, and quantifying, the extent of pathway modulation in any cell-based experiment; and (3) rapid and accurate methods for using gene annotation technologies to map novel genes into biochemical pathways in living cells and to validate novel pharmaceutical targets.

Novel pharmaceutical targets are those proteins which play an active role in a disease-related pathway, that is, a pathway that is associated with or perturbed in the disease state, for which either there are agents with known therapeutic efficacy or for which chemical inhibitors can be developed. Gene or protein silencing, inactivation, or activation can in principle be associated with any cellular or disease phenotype or biological process of interest. The challenge is then to elucidate the underlying biochemical mechanism, network, or pathway, by which the targeted reagent causes the observed effect.

Traditional biochemical methods are used to assess the efficacy of gene or protein targeting. Such methods include quantification of individual proteins or nucleic acids following cell treatment with an annotation reagent. For example, commonly used biochemical assays involve measurements of messenger RNA levels by Northern blotting and/or RT-PCR. Protein-based measurements have typically involved immunoprecipitation or Western blotting.

Typically these assays require the preparation and analysis of cell lysates following the cell treatment; none of the above methods can be applied to intact cells. Such methods are difficult to scale up or automate and are semi-quantitative at best, and are not readily adapted to large-scale studies or to automated analyses of hundreds or thousands of potential drug targets.

Gene expression profiling has also been used on a large scale to evaluate how the expression of other genes is impacted when a target gene is inhibited by a specific probe or reagent (Chi, J-T., et al., Proc. Natl. Acad. Sci. 100: 6343-6346, 2003). Gene expression profiles for a knockout of a novel gene can be compared to those for knockouts of genes within a known pathway; if the profiles are similar, it can be inferred that the novel gene participates in the pathway.

Transcriptional reporter assays have also been used to assess the effect of gene or protein silencing, inactivation or activation on the transcription of other genes under the influence of a promoter linked to a specific cellular signal. These analyses allow inferences to be drawn regarding the pathway in which a gene of interest might participate. For example, it would be possible to infer that a gene of interest is involved in TNF (tumor necrosis factor-dependent) signaling if knocking out the gene of interest caused a reduction in TNF-dependent transcription. These assays have the advantage of being applied to intact cells and can be scaled for high-throughput, automated systems. However, mapping pathways is not possible with transcription reporter assays except through inference. It is often not possible to determine whether the effect on transcription is a direct vs. an indirect effect of the gene on the pathway of interest or to determine the mechanism by which a particular gene affects transcription.

FIG. 6 provides a scheme for understanding the organization of proteins within a biochemical pathway and specifically a signal transduction pathway, and forms the basis for the present invention. The events within a signaling cascade involve the physical association and dissociation of proteins within complexes, and the movement of proteins from one subcellular compartment (such as the membrane, cytosol, or nucleus) to another compartment. The association and dissociation of proteins leads to activation and inactivation of proteins that effect changes in cell behavior and transcriptional activity. Such activation and inactivation events occur by a variety of biochemical mechanisms that involve the transfer of mass and energy between proteins. These events include, for example, post-translational modifications effected by one protein acting upon another. Such modifications include phosphorylation, dephosphorylation, acetylation, myristylation, farnesylation, geranylation, ubiquitination, and other post-translational modifications of proteins. Other events involve control of protein amount, for example by activation or inactivation of proteasomal degradation, or by chaperones such as heat shock proteins. Activation and inhibition can also occur by physical sequestration of proteins, e.g. by binding to lipid rafts or to protein scaffolds. Over-or under-expression of a gene or its cognate protein will alter the stoichiometry of the individual steps in the pathway and their ability to respond to extracellular signals. Therefore, silencing, inactivating or activating a single protein would in principle modulate signals that are transduced through that protein. Thus we refer to events ‘downstream’ or ‘upstream’ of particular proteins of interest. An event ‘downstream’ of a protein of interest is an event that is modulated by that protein acting in the context of the physical networks of the living cell. In turn, the protein of interest may itself be modulated by events ‘upstream’ of that protein within the networks of the living cell.

Observing induction and inhibition of individual biochemical steps within a signaling cascade as a consequence of gene or protein silencing or modulation would enable target validation in the following ways: (1) Perturbation of protein modifications, protein translocation, and protein-protein interactions in predicted ways by inhibitors and stimulators would provide convincing evidence that the individual proteins do in fact participate in a particular pathway; (2) The way in which the stimulators and inhibitors affect an individual protein or its interactions (the “pharmacological profile”) would provide evidence for the position of the particular targets within the pathway; (3) Signal transduction pathways are hierarchically organized in space, with early events occurring at the inner membrane surface while later events may occur in the cytosol, nucleus or other subcellular compartments. A biologically relevant protein complex must occur at a surface or within a cellular compartment that is consistent with its position within the signaling cascade.

It would be extremely useful to have assays that can not only measure the success of a particular annotation reagent within an experiment, but also its mechanism of action within the interconnected networks. Moreover it would be useful to have a direct readout of the effect that does not require cell lysis. Intact cell assay methods could be combined with phenotypic analyses in the same cell populations, allowing direct and rapid correlations of the cellular phenotype with the biochemical pathway responsible for the phenotype. Ideally, such assays would have the following five characteristics: (1) The assay methods should be quantitative, allowing measurements of the degree of inhibition or activation of a particular protein within a pathway. (2) The assay methods should be direct, allowing mapping of individual proteins directly to their sites of action within the complex networks of the living cell. (3) Fluorescence or luminescence assays are preferred due to their suitability for intact cell measurements. (4) The assay methods should be useful on a genome-wide scale, not requiring a custom assay for each class of targets. (5) The assay methods should be compatible with existing high-throughput or high-content instrumentation and formats, such as microtiter plate instrumentation and automated imaging systems.

We sought to identify assays that can be used in conjunction with annotation reagents on such a scale. Here, we describe a strategy and proof of principle for the use of intact cell assays in conjunction with annotation reagents for annotating gene function and mapping biochemical pathways in living cells. In particular we demonstrate a pathway mapping strategy that utilizes gene annotation reagents together with quantitative, high-throughput or high-content assays in intact (live or fixed) cells. The invention allows verification and quantification of the specificity and extent of the effects of any annotation reagent at the level of individual gene products. More importantly, the invention enables mapping genes to their sites of action within biochemical pathways and validating pharmaceutical targets in living cells. In particular, the present invention teaches that biochemical pathways can be mapped by (1) silencing, inactivating or activating individual genes or proteins within living cells, and (2) in the same cells, performing a high-content or high-throughput assay to quantify the amount, subcellular location, or post-translational modification status of proteins within complexes. Importantly, these approaches allow the determination of the effects of an annotation reagent on events ‘downstream’ of the reagent in a biochemical pathway. This enables reconstruction of the hierarchy of biochemical pathways and networks in live cells; an approach not possible with any other methods to date. First, we investigated methods for the dynamic and quantitative measurement of protein-protein interactions in human cells. These methods largely involve the expression or co-expression of genes encoding single or interacting proteins, wherein said proteins are tagged with a reporter or a reporter fragment, and wherein the translocation or association of the proteins in live cells leads to a measurable increase or shift in reporter signal. For example, expressed proteins can be tagged with intact fluorescent proteins such as green fluorescent protein (GFP), and their subcellular redistribution can be followed after a cellular treatment (see for example Schmid, J. A., et al., J. Biol. Chem. 275 (22): 17035-17042, 2000).

If two proteins tagged with different reporters are co-expressed, the interactions of the tagged proteins can be determined. This is the basis for the widely used fluorescence resonance energy transfer (FRET) or bioluminescence resonance energy transfer (BRET) technologies. The resulting shift in emission spectrum upon interaction of the proteins can be quantified.

Mammalian two-hybrid systems have also been developed in which the association of two proteins leads to the expression of a fluorescent protein (U.S. Pat. No. 6,479,289). In addition, fluorescence assays can be constructed by tagging proteins with reporter subunits or fragments. Proteins tagged with complementing enzyme subunits have also been used to construct fluorescence assays in mammalian cells (Rossi, F., Charlton, C. A. & Blau, H. M. Proc. Natl. Acad. Sci. USA 94, 8405-8410, 1997).

In addition, we developed (See U.S. Pat. No. 6,270,964 B1) an experimental approach for detecting biomolecular interactions in living cells based on protein interaction-induced folding and reconstitution of activity of a reporter from two rationally-dissected fragments of an enzyme or a fluorescent, luminescent or phosphorescent protein (see U.S. Pat. No. 6,270,964 B1; see also, Pelletier, J. N., Campbell-Valois, F. & Michnick, S. W., Proc Natl Acad Sci USA 95, 12141-12146(1998); Remy, I. & Michnick, S. W., Proc Natl Acad Sci USA 96, 5394-5399 (1999); Remy, I., Wilson, I. A., and Michnick S. W., Science 283, 990-993(1999); and Michnick, S. W., Remy, I., C.-Valois, F.-X., V.-Belisle, A. & Pelletier, J. N., in Methods in Enzymology (J. N. Abelson, S. D. Emr and J. Thorner, ed) Vol. 328, pp. 208-230, Academic Press, New York (2000)). Generally we call this technology PCA which stands for Protein-fragment Complementation Assays (see Michnick et al, Methods in Enzymology, supra). We have demonstrated that PCAs can be used as high-content or high-throughput assays for the detection, quantitation, localization and activation status of protein-protein complexes and of other biomolecular interactions within living cells.

As discussed above, a biochemical pathway is comprised of a series of steps involving the association and dissociation of proteins within complexes and the exchange of matter and energy. Many such events lead to post-translational modifications of proteins. In principle, silencing of a gene encoding a protein that plays a key role in a signaling pathway would lead not only to a change in the amount or location of proteins ‘downstream’ of the targeted gene, but would result in changes in the post-translational modification status of the same proteins. Given sufficiently sensitive assay methods, such changes could be used in conjunction with gene silencing experiments to unravel the hierarchy of any biochemical pathway and to annotate the function of a large number of novel genes in the context of a living cell or organism. To our knowledge, with the exception of our own work (Remy, I. and Michnick, S. W. Visualization of Biochemical Networks in Living Cells, Proc Natl Acad Sci USA, 98: 7678-7683, 2001) the prior art is silent on large scale methods for annotating gene function and mapping biochemical pathways directly within intact, living cells.

### OBJECTS AND ADVANTAGES OF THE INVENTION

It is an object of the present invention to provide methods for mapping genes and proteins into biochemical pathways, and for validating novel pharmaceutical targets.

It is an additional object of the present invention to provide methods for directly measuring the effects of an annotation reagent on any protein or pathway in an intact cell.

It is an additional object of the present invention to show that the methods provided herein can be applied to any target class, cell type, species, disease mechanism, or organism.

Another object of the invention is to provide methods for specific high-content or high-throughput assays suitable for use in conjunction with a gene annotation technology or gene annotation reagent.

It is a further object of the invention to provide quantitative methods for identifying and validating pharmaceutical targets using gene annotation reagents in combination with such assays.

It is a further object of the invention to provide assay methods, formats, and compositions suitable for these applications on a genome-wide scale.

The invention has the advantage of being applicable on a genome-wide scale. The invention has the further advantage of being applicable to existing laboratory automation and high-throughput instrumentation systems. Moreover, the invention has the advantage of allowing the identification and validation of a large number of new and useful pharmaceutical targets arising from the human genome.

### SUMMARY OF THE INVENTION

The present invention provides quantitative methods for detecting gene or protein silencing, inactivation or activation in intact cells. In particular, the present invention provides that functional annotation of genes, and in particular mapping of gene products into biochemical pathways, can be accomplished using an annotation reagent in combination with a high-throughput or high-content assay in an intact cell.

Importantly, we show that proteins in biochemical pathways can be shown to be functionally linked to other proteins within the same pathways by targeting (silencing, inactivating, or activating) one of the proteins in the pathway of interest with an annotation reagent, such as a gene silencing reagent in a living cell; measuring the effects of the annotation reagent on test proteins of interest in the same cells; and, if the annotation reagent results in modulation of the test proteins in a coordinated manner, identifying the test proteins as being functionally linked to the target of the annotation reagent.

Moreover, we show that a variety of high-throughput and high-content assays in intact cells can be used to identify the dynamic modulations of proteins within pathways in living cells and to quantify the effects of annotation reagents on those pathways.

The invention enables quantification of the success of silencing of a targeted gene. However, the invention goes beyond providing direct assays of gene silencing of targeted genes. The demonstration that entire pathways can be perturbed in a coordinated fashion by a gene silencing reagent or other annotation reagent allows, for the first time, the thousands of proteins encoded by the human genome to be mapped into their functional networks in living human cells. Moreover, the organization and hierarchy of these pathways can be established using these methods, on a scale not possible previously.

The present invention provides for a method of annotating the function of a gene or a protein, said method comprising (a) Contacting a first population of cells with a test reagent, wherein said test reagent is targeted to a particular gene or protein; (b) Measuring the quantity, sub-cellular location, activity, and/or post-translational modification status of a protein or a protein-protein complex of interest in the first population of cells; (c) Measuring the quantity, sub-cellular location, activity and/or post-translational modification status of said protein or protein-protein complex in a second population of cells which has been contacted either with a control reagent or with no reagent; (d) Comparing the results obtained from the first population of cells to the results obtained from the second population of cells; (e) Using the results of step (d) to identify changes in said protein or protein-protein complex caused by said test reagent; and (f) Identifying said protein(s) as being functionally linked to the gene or protein targeted by said test reagent, if said test reagent causes a measurable change in said protein(s).

2) A method of measuring the effects of a test reagent in a living cell, said method comprising (a) Contacting a first population of cells with a test reagent, wherein said test reagent is targeted to a particular gene or protein; (b) Measuring the quantity, sub-cellular location, activity, and/or post-translational modification status of a protein or a protein-protein complex in said first population of cells; (c) Measuring the quantity, sub-cellular location, activity and/or post-translational modification status of said protein or protein-protein complex in a second population of cells which has been contacted either with a control reagent or with no reagent; (d) Comparing the results obtained from said first population of cells to the results obtained from said second population of cells; (e) Using the results of step d to identify changes in said protein or protein-protein complex caused by said test reagent; and (f) Identifying said test reagent as affecting said protein(s) if said test reagent causes a measurable change in said protein(s).

The invention also provides a method of identifying the site of action of a protein within a biochemical pathway, comprising: (a) constructing a high-content or a high-throughput assay for a first protein of interest; (b) constructing a high-content or a high-throughput assay for one or more second proteins within a biochemical pathway or pathways; (c) performing the assays from steps (a) and (b) in the absence and presence of one or more test reagents; (d) using the results of (c) to establish quantitative pharmacological profiles for each of said protein(s); (e) comparing the pharmacological profile for said first protein to the pharmacological profiles for each of said second proteins(s); (f) using the result of (e) to identify the biochemical pathways(s) in which the first protein participates.

The invention further provides a method of identifying or validating novel disease pathways and/or therapeutic targets, said method comprising: (a) Contacting a cell, tissue, or organism with one or more test reagents; (b) Determining the biochemical, biological, phenotypic, and/or physiological effects of each of said test reagents; (c) Based on the results of (b), identifying a test reagent with desired effects; (d) Using a method provided herein, identifying the biochemical pathway(s) in which the target of said reagent participates.

The invention also provides a method for constructing an assay, said method comprising: (a) selecting genes encoding proteins that interact; (b) selecting appropriate reporters or reporter fragments; (c) fusing or attaching said reporters or reporter fragments separately to the genes encoding said interacting proteins; (d) expressing said proteins in living cells; (e) associating said reporters or reporter fragments through interactions of said proteins that are fused or attached to said reporters or said fragments; and (f) measuring the activity of said reporters in the absence or presence of a test reagent.

The invention further provides a method for identifying or validating novel pharmaceutical targets comprising: (a) using a protein-protein interaction assay to identify a first protein that interacts with other proteins within a biochemical pathway of interest; (b) determining whether said protein actively participates in said pathway, by establishing a pharmacological profile for said interaction and comparing said pharmacological profile with the pharmacological profiles of other interactions in the same pathway.

The invention further provides assays for use in conjunction with gene annotation, pathway mapping, and target validation in intact cells.

Important advancements accomplished by the invention, and which are described in fuller detail below, are the ability to annotate the function of a novel gene using gene silencing and to definitely place (or not place) that gene on the ladder of the particular biochemical pathway in which it is believed to participate; the ability to map entire biochemical pathways efficiently and on a large scale using annotation technologies and reagents coupled to a high-content or high-throughput assay; and provision of new high-throughput methodology(ies) for the identification of new pharmaceutical targets.

### DESCRIPTION OF THE FIGURES

FIG. 1 illustrates several components of the AKT signaling pathway used in the instant invention.

FIG. 2 illustrates high-throughput imaging of the fluorescence generated by the AKT/PDK1 protein-protein complex in living cells.

FIG. 3 illustrates the effect of gene silencing with AKT-siRNA on the amount of the AKT/PDK1 protein-protein complex in living cells, and the dose dependence on siRNA.

FIG. 4 illustrates the effect of gene silencing with AKT-siRNA on the amount of the downstream BAD/BCL-xL protein complex in living cells, and the dose dependence on AKT siRNA.

FIG. 5 illustrates the effect of gene silencing with AKT-siRNA on the amount of the downstream BCL-xL/14-3-3sigma protein complex in living cells, and the dose dependence on AKT siRNA.

FIG. 6 illustrates a preferred embodiment of the invention for generating a functional validation profile of a biochemical network and its component proteins or targets.

FIG. 7 shows an entire 96-well plate view of a cell-based protein-fragment complementation assay for AKT1/HSP90 protein complexes, showing the effects of various gene silencing reagents (siRNAs) on the AKT/HSP90 protein complex as assessed by automated fluorescence microscopy. When silenced, genes that participate in the pathway leading to AKT/HSP90 will change the amount or sub-cellular location of the AKT/HSP90 complex within the cells. Examples of increases and decreases in AKT/HSP90 are shown.

FIG. 8 shows the effects of an siRNA targeted against CDC37 on 32 different protein-protein complexes, demonstrating the universality of the approach. Silencing CDC37 leads to changes in the amount or the sub-cellular locations of protein-protein complexes within pathways linked to, or influenced by, CDC37.

FIG. 9 demonstrates the application of the invention to alternative annotation technologies such as the overexpression of a mutationally active protein. Expression of a mutationally activated H-Ras protein (“Ras12V”) leads to an increase in quantity of JNK2/c-Jun protein-protein complexes (FIG. 9A) and in MEK/ERK protein-protein complexes (FIG. 9B) in human cells. Subcellular locations of the respective complexes can also be determined.

FIG. 10 shows the application of the invention to high-content screening systems. In TNF-responsive (HEK293) cells, treatment with TNF leads to a redistribution of the NFkappaB (p65/p50) complex from the cytosol to the nucleus (FIG. 10A). Pretreatment with an siRNA targeted against the TNF receptor (TNFR1 siRNA) abrogates the effect of TNF, causing the p65/p50 complex to remain in the cytosol (FIG. 10B, lower panel). Results with the TNFR1 siRNA are compared to the results of a control (siLUC) reagent (FIG. 10B, upper panel) which had no effect on the assay.

FIG. 11 shows the further application of the invention to high-content assays, wherein the post-translational modification status of proteins is determined by immunofluorescence.

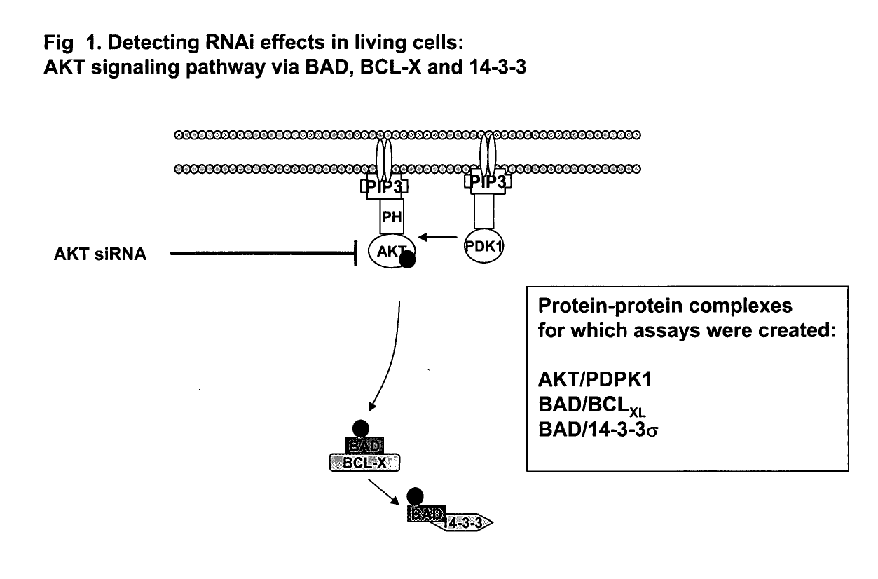

### DETAILED DESCRIPTION OF THE INVENTION

The subject of this invention is a methodology that allows the functional annotation of genes and proteins, mapping of biochemical and disease pathways, and validation of pharmaceutical targets. In particular the present invention provides a strategy for genome-wide mapping of biochemical pathways using an annotation reagent in conjunction with a suitable assay in an intact cell. Such assays may be constructed with or without prior knowledge of the function of the gene or protein of interest. For example, such a strategy may entail first a screening step; a simple assay to detect protein-protein interactions among potential partner proteins, followed by the generation of a functional validation profile. Such a profile would consist of two types of data. First, a biochemical network of interest should be perturbed by specific stimuli or inhibitors. By the same reasoning, the activities, post-translational modifications, and interactions between component proteins of the pathway should be perturbed by these reagents and a pattern of responses or “pharmacological profile” observed that is consistent with the response of the pathway or network studied. A rationale for the approach is described here.

Cellular phenotypes and responses are mediated by a complex array of proteins that are resident within subcellular compartments. The particularly cellular protein architecture is controlled by regulatory elements within the encoded genetic information within the cell nucleus. Binding of proteins, amino acids, growth factors, hormones and other external stimuli such as UV irradiation or oxidative stress induce a cascade of intracellular interactions mediated initially by receptors or other signaling molecules within or associated with the cell membrane or in the cytoplasm. The regulation of cellular responses is mediated by a certain “threshold” number of molecular interactions that eventually reach the cell nucleus and induce the genetic apparatus of the cell to be mobilized to synthesize newly expressed gene products (proteins) in response to the initial stimuli. Gene expression, protein expression and gene silencing technologies have proven particularly powerful in correlating the effects of a specific gene or protein on a cellular phenotype, and developing inferences regarding the mechanism by which the gene or protein induces the cellular phenotype. The present invention enables direct testing of these inferences.

Cell proliferation, cell-death (apoptosis), chemotaxis, metastases etc. are all controlled at the level of the proteins involved, their activities, and their interactions with other proteins. There is no prior art that demonstrates pathway mapping and target validation in intact cells in conjunction with annotation reagents.

The novel methodology of this invention enables: (1) Direct visualization of the molecular architecture of specific cellular responses at the level of the individual proteins and protein interactions that enable such cellular architecture; (2) Direct and quantitative analysis of gene or protein silencing, inactivation, or activation on cellular signaling networks on a scale never previously possible; and (3) The creation of quantitative “Pharmacological Profiles” of proteins and protein-protein interactions induced in response to stimuli throughout the entire cell; from membrane to nucleus.

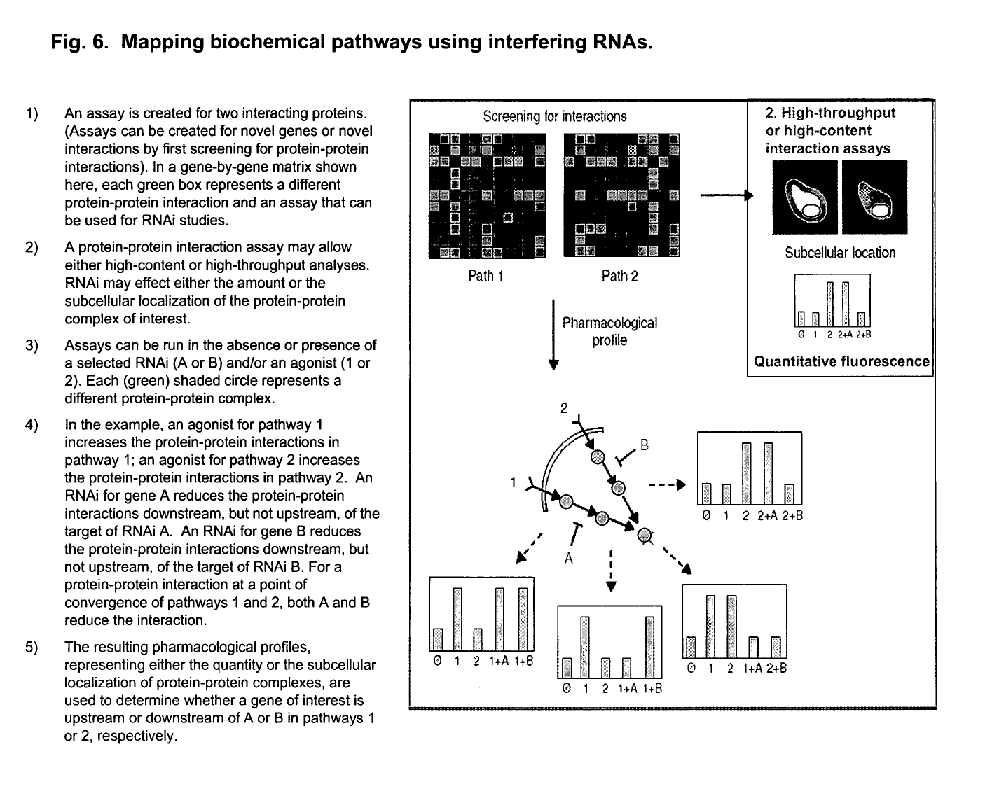

FIG. 6 illustrates a schematic representation of one embodiment of the strategy for generating a functional validation profile of a biochemical network and its component proteins or targets. The figure illustrates a preferred embodiment of the invention for identifying and validating pharmaceutical targets, starting with any gene or genes of interest. In this embodiment the assays are based on protein-protein interactions. The interactions between the product of a gene of interest and the members of one or more pathways are depicted as a gene-by-gene matrix (in practice, screening for such interactions can be performed using a gene-by-gene or a gene-by-library approach where the library is a cDNA library). Positive interactions between gene products are shown in green; lack of an interaction is depicted in red. Proteins interacting with other members of a pathway are then validated by constructing quantitative pharmacological profiles specific to the pathway in question, which is achieved by measuring the effects of perturbants (activators and inhibitors)-including gene silencing reagents on the protein-protein complexes, and by determining the subcellular localization of the complexes e.g. using PCA. Proteins that actively participate in disease-related pathways are potential new targets for discovery of small-molecule, natural product or other potential drug molecules.

The details of the methodology are as follows. Interacting proteins are detected with any one of a large number of protein-protein interaction technologies or screens, which could include enzyme-fragment or protein-fragment complementation assays (PCA); resonance energy transfer assays (FRET or BRET); two-hybrid assays (such as a mammalian two-hybrid assay); split ubiquitin assays; subunit complementation assays (such as a beta-galactosidase complementation assay); or other assays that allow detection of protein-protein complexes. In the example, the expressed proteins correspond to interacting component proteins of two convergent signal transduction pathways (Path 1 and Path 2). An interaction matrix (upper left) represents all positive (green) and negative (red) interacting pairs observed in an interaction screen. The resulting assays, which may be suitable either for high-content or high-throughput quantitative analysis, are subjected to two functional analyses: 1) interactions are probed with pathway-specific stimulators (1 and 2) and gene silencing reagents or other inhibitors (A and B). Pharmacological profiles are established based on the pattern of response of individual interactions to stimulators and inhibitors, represented in the histograms (ordinate axis represent fluorescence intensity). For example, stimulation of pathway 1 will augment all the interactions composing that pathway. The gene silencing reagent A will inhibit protein interactions downstream, but not upstream of its site of action in pathway 1.

In one embodiment of the invention, gene(s) encoding the proteins that encompass a particular signaling pathway of interest are used; preferably as characterized full-length cDNA(s) or less preferably as cDNA libraries. The methodology is not limited, however, to full-length clones and partial cDNA's can also be employed. The methodology is also not limited to overexpression of proteins. Endogenous proteins can also be studied using methods suitable for the measurement of complexes between endogenous proteins. For example, such proteins can be internally tagged—using homologous or non-homologous recombination methods that are well known to those skilled in the art—for use with protein-fragment complementation assays or similar assays. Finally, protein-based targeting can be used to directly modulate the amount or activity of the proteins involved in the pathway, for example by protein overexpression or targeted protein degradation as compared with gene overexpression or gene silencing.

For transient or stable assays based on protein-protein interactions, one of the approaches is as follows. For the signaling network to be studied each of the members of selected cDNAs is cloned as a gene fusion in frame (3′ or 5′) with a suitable reporter, such as a PCA reporter fragment or an intact fluorescent protein, into an appropriate vector such that upon transfection into an appropriate cell line the encoded protein that is subsequently expressed in the cell carries a reporter or reporter fragment either at the amino or carboxy terminus of the protein encoded in the original cDNA selected.

Each member of the signaling network is transfected in a gene-by-gene array or matrix, i.e.:

| | | | | | |
|---|---|---|---|---|---|
| Gene A | Gene B | Gene C | Gene D | Gene E | Etc. |

Gene A
Gene B
Gene C
Gene D
Gene E
Etc.

The results of such an interaction map can be depicted as a gene-by-gene matrix as shown in FIG. 6. The preferred reporters for this approach are any reporters generating a fluorescent signal, since cell-based fluorescent assays are readily performed using off-the-shelf instrumentation for fluorescence quantitation and/or imaging. Suitable PCA reporters include a variety of fluorescent proteins (GFP, YFP and mutants thereof); DHFR; beta-lactamase; Renilla luciferase; and other reporters previously described. Suitable reporters for FRET or BRET have been described in the literature.

Quantification of the number of protein-protein complexes for each pairwise interaction in the cell is achieved by fluorescence detection in a fluorescence microtiter plate reader and/or by imaging the subcellular location of the protein-protein complex by microscopy or automated image analysis. Stimulation and inhibition of the interactions by a gene annotation reagent, such as by siRNA or vector-based expression of native or mutant proteins, or by polypeptide reagents, is monitored after treatment of the live cells with the reagent. This allows the profiling of responses for each interacting pair of the pathway and also allows the site of intervention of agonists/antagonists within each pathway as well as locating cross-talk points between different signaling pathways as embodied in the attached publication.

A general description of the methodology is as follows. Discrete populations of cells (for example, in wells of a multi-well tissue culture plate) are treated with an agent that modulates the cellular activity. This can be achieved by transfecting or infecting or contacting or injecting cells with the desired annotation reagents. As described above, suitable annotation reagents may include wild-type, dominant negative or activated, siRNA or antisense gene expression vectors, or isolated siRNAs, “decoy” DNAs, proteins, or polypeptides. Alternatively, cells can be microinjected with cDNAs, proteins, or peptides; or treated with transduction reagents (e.g. TAT or CPIP-fusion polypeptides. Additionally, reagents can be used which do not enter the cell, but act via binding to cell surface receptors. Following treatment with the annotation reagent, cells are cultured for a variable length of time. Cells are then subjected to microscopic imaging or signal intensity measurements. Specific cellular activities (protein complex formation, protein translocation, changes in protein levels or post-translational modification) are visualized by various assay methods. Optimally such methods involve high-content assays or high-throughput assays with intact cells following treatment of the cells with an annotation reagent. The effects of the annotation reagent are determined by comparison of the assay results with the results from untreated cells, or from cells treated with a control reagent.

In making the invention we show that high-throughput and/or high-content assays in intact cells can be used to quantify the extent of silencing by measuring a reduction in the amount of protein-protein complex formed between the protein encoded by the targeted gene and a cognate binding partner. Thus these assays serve as direct indicators of gene silencing. More important for purposes of pathway mapping and target validation, we show for the first time that assays for proteins downstream of a silenced gene can be used to link the downstream proteins to the product of the gene that is silenced. Therefore the present invention enables the hierarchy of biochemical pathways to be established, an achievement which has never before been demonstrated in conjunction with gene silencing and that will be extremely useful for the validation of new pharmaceutical targets.

In the first set of examples, protein pairs previously identified by PCA as forming complexes in human cells were co-transfected with either a targeted siRNA (siAKT) or a non-targeted siRNA (negative control) and the two populations of cells were monitored for loss of fluorescence intensity. In the second set of examples, the pathways leading to the interaction of AKT with HSP90beta (heat shock protein 90) were investigated, by testing the ability of a plurality of different siRNAs to enhance or diminish the formation or subcellular locations of complexes between AKT and HSP90. In the third set of examples, the role of a specific protein—CDC37—within biochemical pathways was examined by testing the ability of siCDC37 to enhance or diminish the formation of 32 distinct protein-protein complexes. In the fourth set of examples, the principles of the invention were extended to other genetic manipulations, by demonstrating the ability of a mutant, activated form of the H-Ras oncogene to activate signaling pathways downstream of H-Ras. In the fifth example the effect of silencing a receptor on the functions of proteins downstream of the receptor in a signaling pathway are demonstrated, with the TNF-dependent pathway as the example. This example also illustrates the use of high-content assays in conjunction with the invention. In the final example an alternative high-content assay is used in which the post-translational modification status of proteins serves as an indication of silencing of upstream elements in a biochemical pathway.

### EXAMPLES

The present invention is illustrated by the following Examples, but should not be construed to be limited thereto.

The decision between survival and death is an important aspect of cellular regulation during development and malignancy. Central to this regulation is the process of programmed cell death or apoptosis. A variety of signaling cascades have been implicated in modulation of apoptosis including the phosphatidylinositol 3′ kinase (PI3′K) pathway. Activation of PI3′K, a lipid kinase, protects against apoptosis, whereas its inhibition enhances apoptosis. The protective effects of PI3′K in mammalian cells have been linked to activation of a series of phosphatidylinositol-dependent kinases (PDK's), including PDK-1, PDK-2 and protein kinase B (PKB or AKT). AKT is a mitogen-regulated protein kinase involved in the protection of cells from apoptosis, the promotion of cell proliferation and diverse metabolic responses. (For a review of AKT and its biochemical functions and binding partners, see Brazil D. P., Park J., and Hemmings B. A., Cell 111:293-303, 2002). The activation of AKT by PI3′K is mediated through the binding of phosphatidylinositol phospholipids to the pleckstrin homology (PH) domain of AKT. Binding of the PH domain of AKT to membrane phosphatidylinositol phospholipids causes the translocation of PKB to the plasma membrane bringing it into contact with membrane-bound AKT-kinases [phosphatidylinositol-dependent kinase-1 and -2 (PDK1 and 2)], which phosphorylate and activate AKT (Thr 308 and Ser 473). AKT is a proto-oncogene that inhibits apoptosis by phosphorylating a number of downstream targets. This includes phosphorylation of Bad (Ser112 and Ser136): The phosphorylation of the pro-apoptotic protein Bad results in its cytosolic sequestration by 14-3-3 proteins which prevents the binding of Bad to the cell survival factor Bcl-xL at intracellular membrane sites. Thus, inhibition of AKT activation induces cancer cell apoptosis. AKT is thought to be involved in cell survival via interaction with known members of the BCL-2 family of proteins (figure x; reviewed in Datta et al., 1999, Cellular survival: a play in three Akts. Genes and Development 13, 2903-2927). BAD, a pro-apoptotic member of the BCL-2 family is a direct substrate of AKT. BAD protein binds anti-apoptotic proteins (BCL-2 and BCL-xL) to promote apoptosis. Phosphorylation of BAD at either of two serine sites results in creation of a consensus binding site for the 14-3-3 protein, triggering the dissociation of BAD from BCL-xL and sequestering BAD in the cytoplasm where it is unable to promote apoptosis (reviewed in Downward, J. Nature Cell Biology 1: E33-E35, 1999; Hirai, I. and H. Wang, Biochem. J. 359: 345-352, 2001).

It has been reported that AKT is overexpressed in some mammalian pancreatic, prostate, ovarian and breast cancers as well as glioblastomas and leukemia. It is also known that defects in the insulin signaling pathway are associated with mutations in AKT. The availability of cell-based assays to identify novel elements and modulators of such a signaling pathway could lead to the identification of novel therapeutic targets, and new pharmaceutical agents that could be effective in treating a number of diseases associated with dysregulation of PI3K-AKT signaling. In addition to cancer, such diseases may include hyperglycemia, non-insulin dependent diabetes mellitus (NIDDM), obesity, hypertension, hypercholesterolemia, hypertriglyceremia, and related disorders.

We used PCA to demonstrate the strategy for mapping interactions, detect the success of gene silencing by RNA interference (RNAi) using small interfering siRNAs, and to begin the process of constructing a map of the hierarchy of proteins within the P13K-regulated AKT pathway. By targeting regulatory proteins in a specific pathway, we demonstrate that both the target proteins and downstream functional complexes and their associated elements are significantly effected.

### Example 1

### DNA Constructs

The full-length cDNAs encoding PDPK1, AKT1, BAD, BCL-xL and 14-3-3 sigma (all of which are known proteins and the corresponding full-length sequence of each is known in the art) were amplified by PCR and subcloned into a mammalian expression vector (pcDNA3.1 Z, Invitrogen) which had been previously modified to contain either fragment 1 (Y1) of a yellow variant of E. victoria GFP (EYFP-F 1, corresponding to GFP amino acids 1 to 158; the protein and fragment sequences being previously disclosed in commonly assigned pending U.S. application Ser. No. 10/772,021 filed Feb. 5, 2004) or fragment 2 (Y2) of EYFP (EYFP-F2, corresponding to GFP amino acids 159 to 239; the protein and fragment sequences being previously disclosed in commonly assigned pending U.S. application Ser. No. 10/724,178 filed Dec. 1, 2003). In all constructs, a 10 amino acid flexible linker consisting of glycine and serine residues [(Gly. Gly. Gly. Gly. Ser)₂] such as that previously disclosed and employed in the invention in commonly assigned U.S. Pat. No. 6,270,964 and application Ser. No. 10/724,178 (pending) was also inserted between the cDNA and the YFP fragments to ensure that the orientation/arrangement of the fusions in space is optimal to bring the EYFP protein fragments into close proximity. AKT1 was fused 3′ of the Y1 fragment, while BAD was fused 3′ of Y2 fragment. BCL-xL and 14-3-3σ were fused 5′ of the Y1 fragment, and PDPK1 was fused 5′ of the Y2 fragment.

### Interfering RNA

SMARTpool AKT1 NM_{—}005163 (siAKT; Dharmacon RNA Technologies, Inc.) 5 nmoles pellet was resuspended in 250 microliters of RNase free water to yield a 20 micromolar stock solution as per the manufacturers instructions. For a control, the luciferase GL2 Duplex (siLUC; Dharmacon RNA Technologies, Inc.) (5 nMoles) was resuspended in 250 microliters of RNase free water to yield a 20 micromolar stock solution per the manufacturers instructions. Stock solutions were aliqouted and stored at −20° C. per the manufacturers specifications for solution storage, and were used in experiments for <3 freeze-thaw cycles.

### Cell Lines and Transfections

HEK293T cells were grown in DMEM (Life Technologies) supplemented with 10% fetal bovine serum (FBS, Hyclone) and 1× Penicillin-Streptomycin (Life Technologies). Twenty four hours prior to transfection, HEK293T cells were plated in 96 well clear bottom black sided tissue culture dishes (Greiner) at a density of 1.5×10⁴ total cells/100 microliters/well. Cells at a confluence of approximately 60% were cotransfected with a total of 0.1 micrograms DNA/well (0.05 micrograms of each pair) and 0.1 or 0.2 microliters of siRNA stock (0.002 or 0.004 nMoles final). PCA pairs consisted of Y1-AKT1 interacting with PDPK1-Y2, Y2-BAD interacting with BCL-xL-Y1, and Y2-BAD interacting with 14-3-3□-Y1. Each PCA pair was cotransfected with either siAKT or control siLUC using Lipofectamine 2000 transfection reagent (Life Technologies) according to the manufacturers recommendations. Transfected cells were incubated at 37° C. in a 0.5% CO² atmosphere for 48 hours. Cells were then washed once with PBS (Life Technologies). The relative amount of reconstituted YFP fluorescence due to the interaction of the PCA pairs was detected by fluorimetric analysis in intact HEK293T cells using a Spectra Max Gemini EM (Molecular Devices; excitation: 485; emission: 527). Post analysis, all wells were stained with 5-10 micromolar DRAQ5 nuclear stain (Biostatus Limited) and then imaged using a Discovery-1 Cell Based Screening System (Universal Imaging Corporation) using a YFP filter set (exciter: 480/40; emitter: 535/50; beamsplitter: Q505lp) for yellow fluorescence and a Cy5 filter set (exciter: 615/30; emitter: 700/60; beamsplitter: 680lp) for red fluorescence.

### Results

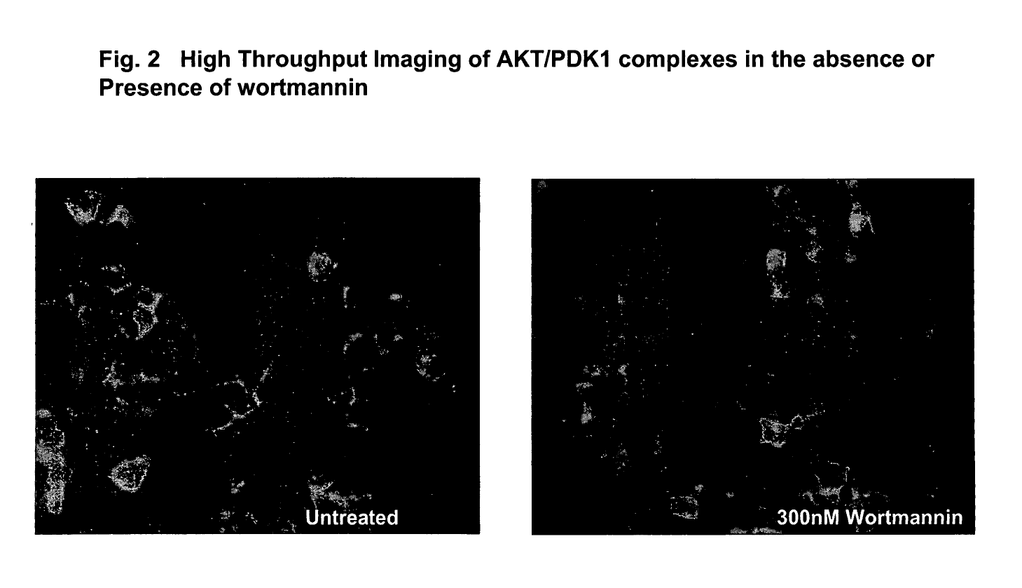

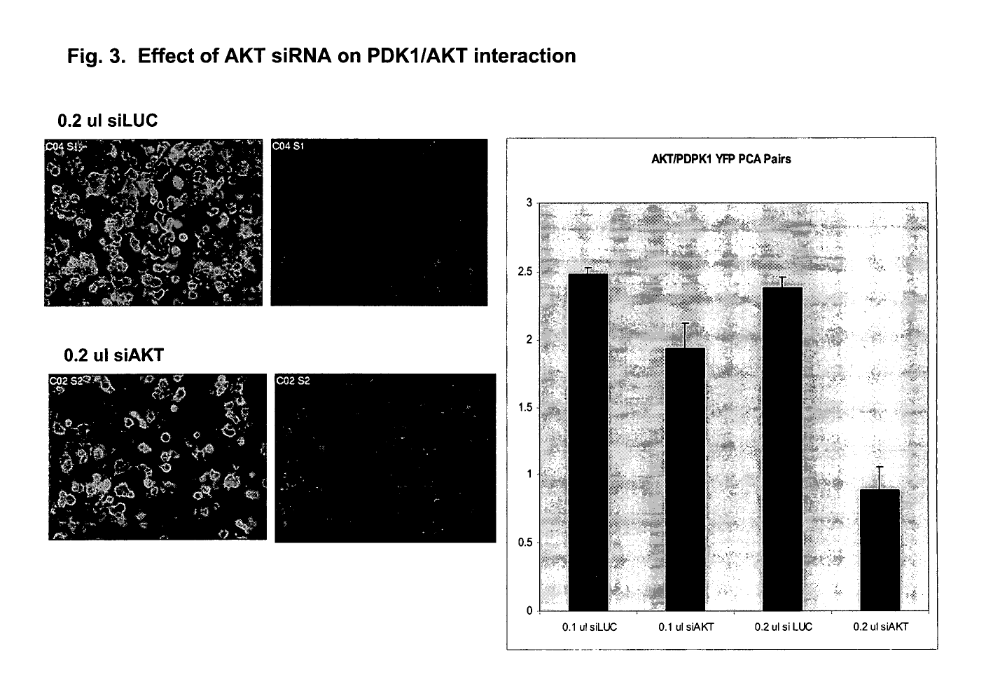

The AKT1 interaction with PDK1 resulted in a reconstitution of YFP fluorescence signal that was predominately membrane localized in activated cells (FIG. 2). The complex is sensitive to pathway modulation, for example by the drug wortmannin which blocks P13 kinase and prevents membrane localization of the AKT/PDPK1 complex (FIG. 2).

The effect of AKT siRNA on the AKT/PDPK1 complex was examined to determine if silencing of AKT would result in a decrease in the protein-protein complex. The localization of AKT+PDPK1 was not affected by cotransfection of this pair with siLUC. Co-transfection of AKT+PDPK1 with siAKT resulted in a significantly decreased signal relative to that seen with siLUC cotransfected pairs that was dose dependent (FIG. 3), demonstrating that the target vector of siAKT, the AKT protein, is directly affected by this RNAi. A dose response was also observed, with 0.2 ng siRNA giving a greater reduction in fluorescence than for 0.1 ng siRNA.

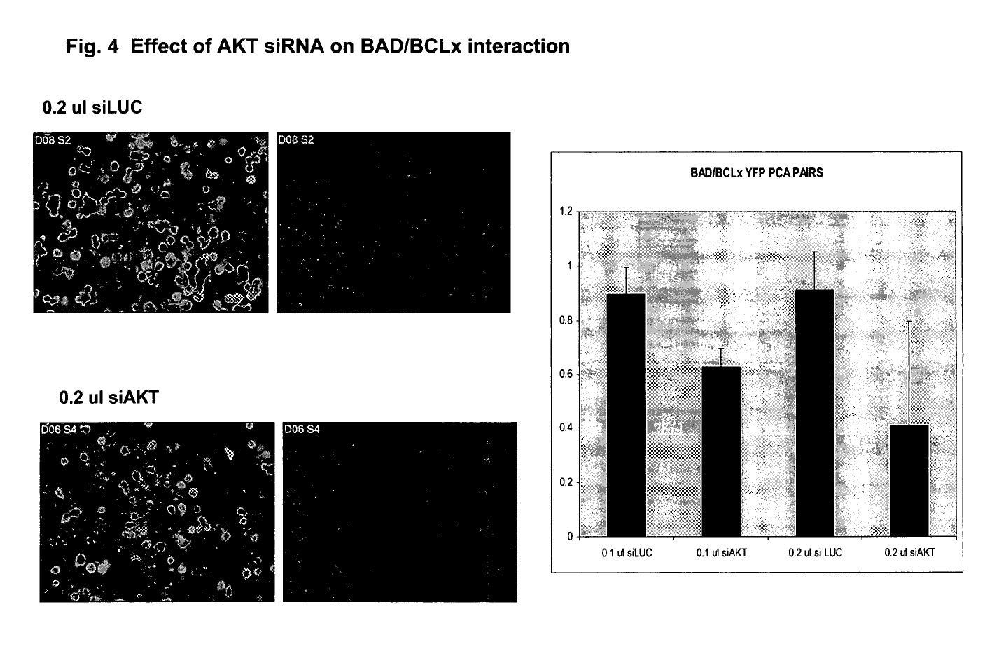

The Y2-BAD interaction with BCL-xL-Y1 also resulted in reconstitution of YFP fluorescence signal that was predominately localized to the cytosol (FIG. 4). Sub-cellular localization of the BAD+BCL-xL complex was not affected by cotransfection with the control reagent, siLUC. It might be expected that inhibition of the AKT phosphorylation of serine site 136 on BAD by silencing of AKT would result in an increase in BAD/BCL-xL complexes due to the inability of BAD to disassociate and translocate from the mitochondria. However, BAD is also phosphorylated at ser 112 by an AKT-independent mechanism, and phosphorylation of either site is sufficient for release of BAD from BAD/BCL-xL complexes and subsequent translocation to the cytosol for binding to 14-3-3 protein. Furthermore, it has been shown that colony stimulating factor (CSF)-induced phosphorylation of BAD is insensitive to P13-Kinase inhibitors in some cell types, strongly suggesting activation of AKT is not sufficient to inactivate BAD in these cell types. In our experiments, cotransfection of BAD+BCL_{XL} with siAKT resulted in a decrease in fluorescence signal (FIG. 4), suggesting that AKT may play a more complex role in the association and disassociation of BAD+BCL_{XL} complexes than was previously thought.

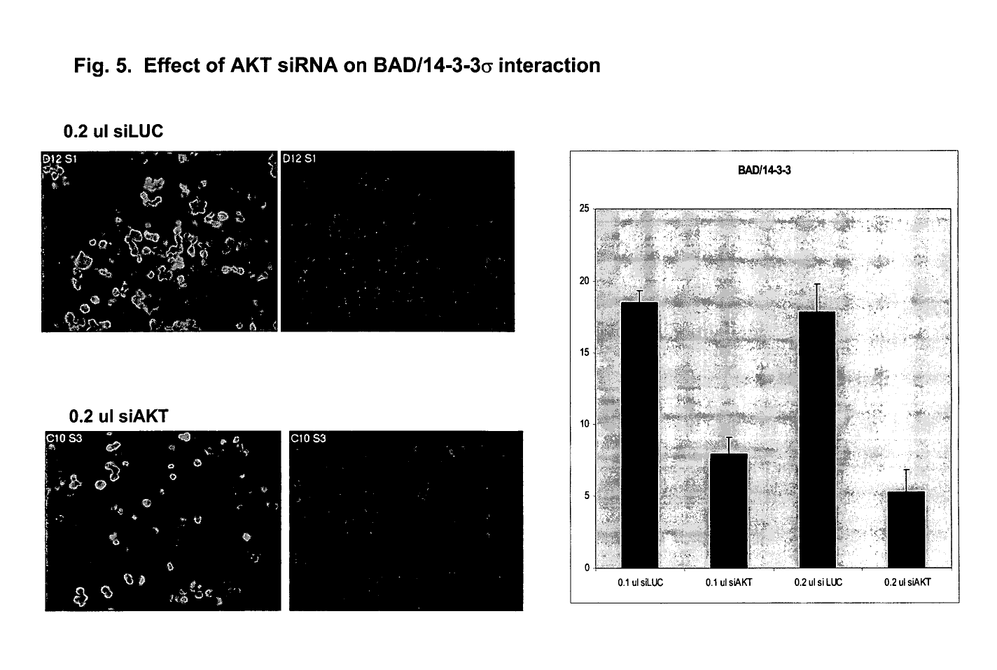

The BAD interaction with 14-3-3sigma resulted in reconstitution of YFP fluorescence with the expected cytosolic localization. Cotransfection of this pair with siLUC did not effect its localization. We tested whether targeted RNAi silencing of AKT would result in failure of BAD to phosphorylate at ser site 136, thus preventing BAD from translocating to the cytosol and forming complexes with 14-3-3sigma. In our experiments, cotransfection of this pair with siAKT resulted in a significant and dramatic reduction in fluorescence signal, strongly suggesting that BAD is no longer available for binding to the 14-3-3sigma protein (FIG. 5).

The use of gene silencing in conjunction with the invention described here can be extended to large-scale mapping of pathways. For example, a single protein-protein complex can be mapped into pathways by studying the effects of silencing of a large number of individual gene targets, one by one, on a particular protein-protein complex. Alternatively, a single target can be placed in the context of other biochemical interactions by studying the effect of silencing that particular target on a large number of protein-protein complexes. Both of these aspects of the invention are further exemplified below. In addition, we show that the invention can be combined with automated high-throughput imaging systems to enable high-throughput pathway mapping on an unprecedented scale.

### Example 2

### Large-Scale Mapping of Biochemical Pathways in High Throughput Formats

In mammalian cells, AKT is protected from degradation and dephosphorylation by binding to a heat shock protein (HSP90) which acts as a chaperone. HSP binding to AKT is thought to occur via the Cdc37 co-chaperone (Brazil et al., 2002). AKT present in protein complexes containing HSP90 and CDC37 was catalytically active and phosphorylated GSK3beta in vitro. HSP90 complex formation with AKT may therefore facilitate kinase activation by preventing both dephosphorylation and AKT degradation. Perturbation of this interaction, for example by ansamycin antibiotics that act as inhibitors of HSP90, or by a expressing a dominant negative form of PKB, leads to inactivation of AKT by dephosphorylation or proteasome-mediated degradation. HSP90 also stabilizes PDK1 in cells, enhancing the phorphorylation of PKB. HSP90 may also act as a scaffold protein, presenting substrates such as eNOS to PKB for phosphorylation. We used this pathway to demonstrate the use of the present invention for large-scale mapping of proteins to biochemical pathways. In particular we sought to demonstrate that silencing of various genes ‘upstream’ of AKT/HSP90 leads to increases or decreases in complex formation, demonstrating functional links between these proteins and other proteins in human cellular networks.

### Construction of PCA Fusion Constructs

Fusion-reporter construct creation and DNA preparation were as described above. Briefly, the full coding sequence for each gene of interest was amplified by PCR from a sequence-verified full-length cDNA and fused to either the 5′ or 3′-end of a yellow fluorescent protein reporter fragment (YFP[1] or YFP[2]) through a linker encoding a flexible 10 amino acid peptide (Gly.Gly.Gly.Gly.Ser)₂. DNAs for reporter fusion constructs were isolated using Qiagen MaxiPrep kits (Qiagen, Chatsworth, Calif.). PCR was used to assess the integrity of each fusion construct, by combining the appropriate gene-specific primer with a reporter-specific primer to confirm that the correct gene-fusion was present and of the correct size with no internal deletions.

### Transient Transfections and PCA Assay Layout

HEK293 cells were maintained in MEM alpha medium (Invitrogen) supplemented with 10% FBS (Gemini Bio-Products), 1% penicillin, and 1% streptomycin, and grown in a 37° C. incubator equilibrated to 5% CO₂. Twenty-four hours prior to transfection, cells were seeded (typically 14,000 cells per well) in 96-well plates coated with poly-D-Lysine (Greiner) using a Multidrop 384 peristaltic pump system (Thermo Electron Corp., Waltham, Mass). A panel of 108 siRNA SMART pools and two ‘GC-matched’ non-specific controls (Dharmacon, Boulder, Colo.) were resuspended in 1× Universal buffer and arrayed into RNase free microtiter plates (manufacturer?) using a Biomek FX (Beckman Coulter, Fullerton, Calif.). The SMART pools used in the study are listed in Table 1. All siRNAs were designed to target human genes.

TABLE 1

| siRNA # | siRNA Name | Protein Target | Gene Accession | Dharmacon Product Number |
|---|---|---|---|---|
| 1 | PTEN | PTEN | NM_000314 | M-003023-00-05 |
| 2 | PIK3CA | p110α PI3K | NM_006218 | M-003018-00-05 |
| 3 | PIK3R1 | p85α PI3K | XM_043865 | M-003020-00-05 |
| 4 | PDPK1 | Pdk1 | NM_002613 | M-003558-00-05 |
| 5 | AKT1 | Akt1 | NM_005163 | M-003000-00-05 |
| 6 | AKT2 | Akt2 | NM_001626 | M-003001-00-05 |
| 7 | GSK3B | Gsk3β | NM_002093 | M-003010-00-05 |
| 8 | RPS6KB1 | p70S6K | NM_003161 | M-003616-00-05 |
| 9 | FRAP1 | FRAP/TOR | NM_004958 | M-003008-01-05 |
| 10 | FKBP | FK506-BP (12 kD) | NM_054014 | M-005183-00-05 |
| 11 | HSPCA | Hsp90α | NM_005348 | M-005186-00-05 |
| 12 | HSPCB | Hsp90β | NM_007355 | M-005187-00-05 |
| 13 | CDC37 | Cdc37 | NM_007065 | M-003231-00-05 |
| 14 | TEBP | p23 | NM_006601 | M-005192-00-05 |
| 15 | BAD | BAD | NM_004322 | M-003870-00-05 |
| 16 | cIAP1 | cIAP1 | NM_001166 | M-004390-00-05 |
| 17 | cIAP2 | cIAP2 | NM_001165 | M-004099-00-05 |
| 18 | Smac/Diablo | Smac/Diablo | NM_019887 | M-004447-00-05 |
| 19 | BCL2 | BCL2 | NM_000633 | M-003307-00-05 |
| 20 | BCL-xL | BCL-xL | NM_138578 | M-003458-00-05 |
| 21 | TNFR1 | TNF-R | NM_001065 | M-005197-00-05 |
| 22 | RIP2 | RIP2 | NM_003821 | M-005370-00-05 |
| 23 | RIP4 | RIP4 | NM_020639 | M-005308-00-05 |
| 24 | TRADD | TRADD | NM_003789 | M-004452-00-05 |
| 25 | FADD | FADD | NM_003824 | M-003800-00-05 |
| 26 | TRAF2 | TRAF2 | NM_021138 | M-005198-00-05 |
| 27 | TRAF6 | TRAF6 | NM_004620 | M-004712-00-05 |
| 28 | CHUK1 | IKKα | NM_001278 | M-003473-00-05 |
| 29 | IKBKB | IKKβ | XM_032491 | M-004120-00-05 |
| 30 | IKBKE | IKKε | NM_014002 | M-003723-00-05 |
| 31 | NFKBIA | IκBα | NM_020529 | M-004765-00-05 |
| 32 | NFKB1B | IκBβ | NM_002503 | M-004764-00-05 |
| 33 | RELA/p65 | NFκB-p65 | NM_021975 | M-003533-00-05 |
| 34 | p50 | NFκB-p50 | NM_003998 | M-003520-00-05 |
| 35 | CREBBP | CBP | NM_004380 | M-003477-00-05 |
| 36 | HDAC1 | HDAC1 | NM_004964 | M-003494-00-05 |
| 37 | HDAC2 | HDAC2 | NM_001527 | M-003495-00-05 |
| 38 | SRC-1 | SRC-1 | U90661.1 | M-005196-00-05 |
| 39 | ESR1 | ERα | NM_000125 | M-003489-00-05 |
| 40 | PPARG | PPARγ | NM_138712 | M-003436-00-05 |
| 41 | RXRA | RXRα | NM_002957 | M-003443-00-05 |
| 42 | SKP2 | Skp2 | NM_005983 | M-003541-00-05 |
| 43 | b-TRCP | β-TRCP | NM_033637 | M-003463-00-05 |
| 44 | MDM2 | Hdm2 | NM_002392 | M-003279-00-05 |
| 45 | TP53 | p53 | NM_000546; M14695 | M-003329-00-05 |
| 46 | ATM | ATM | NM_000051 | M-003201-00-05 |
| 47 | ATR | ATR | NM_001184 | M-003202-01-05 |
| 48 | ABL1 | c-ABL | NM_007313 | M-003100-01-05 |
| 49 | BRCA1 | Brca1 | NM_007295 | M-003461-00-05 |
| 50 | CHEK1 | Chk1 | NM_001274 | M-003255-01-05 |
| 51 | CHEK2 | Chk2 | NM_007194 | M-003256-00-05 |
| 52 | CDC25A | Cdc25A | NM_001789 | M-003226-00-05 |
| 53 | CDC25C | Cdc25C | NM_001790 | M-003228-00-05 |
| 54 | PLK | Plk | NM_005030 | M-003290-00-05 |
| 55 | CDK4 | Cdk4 | NM_000075 | M-003238-00-05 |
| 56 | RB1 | Rb | NM_000321 | M-003296-00-05 |
| 57 | CDKN1A | Cip/p21 | NM_078467; | M-003471-00-05 |
| | | | NM_000389 | |
| 58 | CDKN1B | Kip/p27 | NM_004064 | M-003472-00-05 |
| 59 | p16INK4 | INK4/p16 | NM_000077 | M-005191-00-05 |
| 60 | 14-3-3s | 14-3-3σ | NM_006142 | M-005180-00-05 |
| 61 | STAT1 | Stat1 | NM_007315 | M-003543-00-05 |
| 62 | JAK1 | Jak1 | NM_002227 | M-003145-01-05 |
| 63 | EGFR | EGFR | NM_005228 | M-003114-01-05 |
| 64 | SRC | c-Src | NM_005417 | M-003175-01-05 |
| 65 | GRB2 | Grb2 | NM_002086 | M-004112-00-05 |
| 66 | SOS1 | Sos1 | NM_005633 | M-005194-00-05 |
| 67 | SOS2 | Sos2 | XM_043720 | M-005195-00-05 |
| 68 | PLCG1 | PLC-γ | NM_002660 | M-003559-00-05 |
| 69 | RaIGDS | RaIGDS | NM_006266 | M-005193-00-05 |
| 70 | RAS | Ha-Ras | NM_005343 | M-004142-00-05 |
| 71 | KRAS2 | K-Ras | NM_004985 | M-005069-00-05 |
| 72 | RAF1 | c-Raf | NM_002880 | M-003601-00-05 |
| 73 | B-Raf | B-Raf | NM_004333 | M-003460-00-05 |
| 74 | MEK1 | Mek1 | NM_002755 | M-003571-00-05 |
| 75 | MEK2 | Mek2 | NM_030662 | M-003573-00-05 |
| 76 | ERK2 | Erk2 | M84489 | M-003555-02-05 |
| 77 | MAPK3 | Erk1 | AK091009 | M-003592-00-05 |
| 78 | ELK1 | Elk1 | NM_005229 | M-003885-00-05 |
| 79 | VAV1 | Vav1 | NM_005428 | M-003935-00-05 |
| 80 | CDC42 | Cdc42 | NM_001791 | M-005057-00-05 |
| 81 | RAC1 | Rac1 | NM_018890 | M-003560-00-05 |
| 82 | PAK1 | Pak1 | NM_002576 | M-003521-00-05 |
| 83 | PAK2 | Pak2 | NM_002577 | M-003597-00-05 |
| 84 | PAK3 | Pak3 | AF068864 | M-003614-00-05 |
| 85 | PAK4 | Pak4 | NM_005884 | M-003615-00-05 |
| 86 | RhoA | RhoA | NM_001664 | M-004549-00-05 |
| 87 | ROCK1 | p160-ROCK | NM_005406 | M-003536-00-05 |
| 88 | MAP3K1 | MEKK1 | XM_042066 | M-003575-00-05 |
| 89 | MAP2K7 | MKK7/JNKK2 | NM_005043 | M-004016-00-05 |
| 90 | ASK1 | MEKK5 | E14699 | M-004539-00-05 |
| 91 | MAP2K4 | MKK4/JNKK1 | NM_003010 | M-003574-00-05 |
| 92 | JNK2 | JNK2 | L31951 | M-003766-00-05 |
| 93 | JNK1 | JNK1 | L26318 | M-003765-00-05 |
| 94 | ITGa4 | ITGα4 | L12002 | M-005189-00-05 |
| 95 | PTK2 | FAK | NM_005607 | M-003164-01-05 |
| 96 | CTNNB1 | β-catenin | NM_001904 | M-003482-00-05 |
| 97 | DVL1 | Dsh1 | U46461 | M-004068-00-05 |
| 98 | DVL2 | Dsh2 | NM_004422 | M-004069-00-05 |
| 99 | EDG4 | Edg-4/LPA2 | AF233092 | M-004602-00-05 |
| 100 | EDG7 | Edg-7/LPA3 | NM_012152 | M-004895-00-05 |
| 101 | GNAI3 | Gαi-3 | NM_006496 | M-005184-00-05 |
| 102 | GLUT4 | GLUT4 | NM_001042 | M-005185-00-05 |
| 103 | PP2AB(cat) | PP2CB | NM_004156 | M-003599-00-05 |
| 104 | PPP2CA | PP2CA | NM_002715 | M-003598-00-05 |
| 105 | PKC | PKCα | NM_002737 | M-003523-00-05 |
| 106 | PRKACG | PKA C-γ | NM_002732 | M-004651-00-05 |
| 107 | PRKACB | PKA C-β | NM_002731 | M-004650-00-05 |
| 108 | AKAP | AKAP1/PRKA1 | NM_003488 | M-005181-00-05 |

HEK293 cells were transfected with 100 ng of nucleic acid per well (up to 50 ng of each fusion construct, the appropriate siRNA SMART pool (40 nM final concentration) and carrier DNA as necessary) with Lipofectamine 2000 (Invitrogen) using a Biomek 2000 workstation. All transfections were aliquoted in triplicate such that each assay, containing a single PCA pair, spanned four 96-well plates. Each 96-well plate contained five internal controls: mock transfected, no siRNA, non-specific siRNA controls IX and XI (47% and 36% GC content, respectively; Dharmacon), and a PCA-specific control (to confirm degree of stimulation for assays requiring stimulation with agonists). The list assays used in this study, their agonists, and corresponding gene and reporter fragment information, are listed in Table 2. Assays 24, 27 and 29 were treated with 500 nM camptothecin (CPT; Calbiochem) for 18 hours prior to fixation. Assay 6 was treated with 50 ng/ml TNFalpha (Roche) for 30 minutes, and the PARgamma:SRC-1 (assay 31) was stimulated with 15 micromolar rosiglitazone (LKT Labs, Inc.) for 90 minutes prior to fixation.

The plate layout is as follows. All wells of the 96-well plate, except wells A1-A3 (mock), were co-transfected with DNA encoding the Akt1 and Hsp90 proteins, each fused to complementary fragments of YFP. Twenty-five siRNA SMART pools (Dharmacon) were transfected in triplicate wells, along with two non-specific control siRNAs. Two sets of triplicate wells (B1-B3, H10-H12) served as controls for the PCA signal, in the absence of any siRNA. Forty-eight hours after transfection, cells were simultaneously fixed in 2% formaldehyde (Ted Pella) and stained with 33 micrograms/ml Hoechst 33342 (Molecular Probes) for 10 minutes on a Biomek FX workstation. Cells were subsequently rinsed with HBSS (Invitrogen) and maintained in the same buffer during image acquisition.

Images for four non-overlapping sites per well (12 images per sample) were acquired using a 20× objective on a Discovery-1 automated fluorescence imager in the YFP (excitation filter 480/40 nm, emission filter 535/50 nm) and DAPI (excitation filter 360/40 nm, emission filter 465/30 nm) channels.

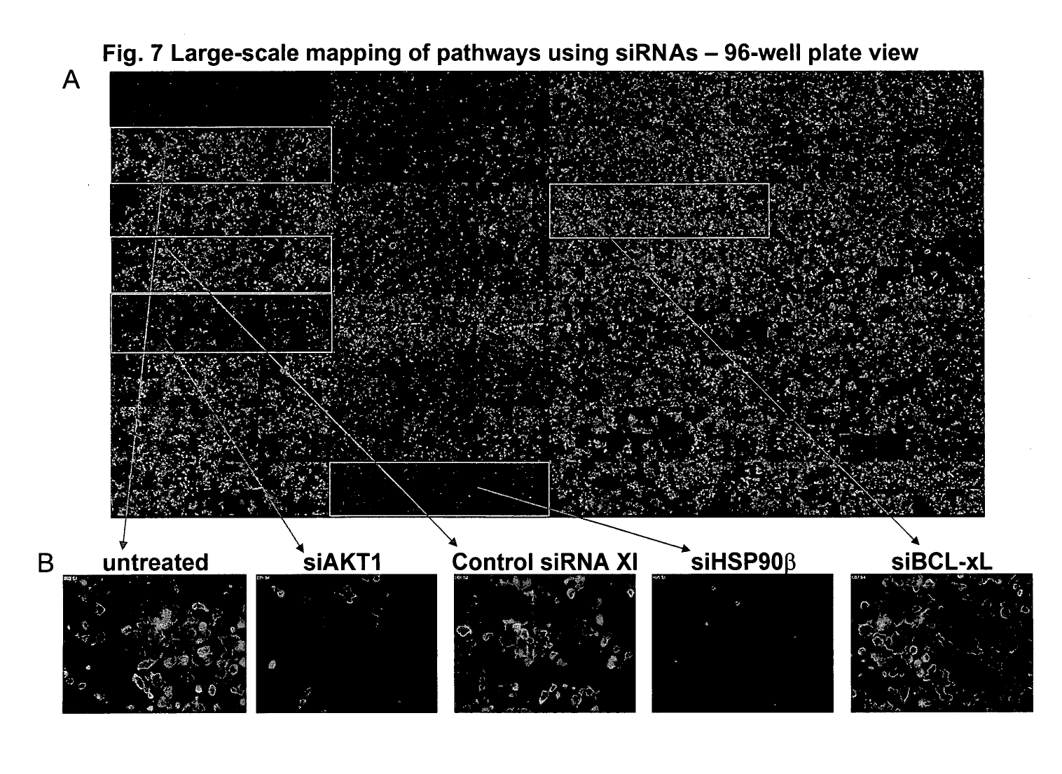

A color composite of images comprising the entire plate (FIG. 7) was generated using Metamorph software (Molecular Devices, Inc.). The siRNAs used are as follows: non-specific control IX (47% GC; C1-C3), non-specific control XI (36% GC; D1-D3), siAKT1 (E1-E3), siAKT2 (F1-F3), siPTEN (G1-G3), siPIK3CA (H1-H3), siGSK3B (A4-A6), siPDPK1(B4-B6), siRPS6 KB1 (C4-C6), siFRAP1 (D4-D6), siFKBP (E4-E6), siCDC37 (F4-F6), siHSPCA (G4-G6), siHSPCB (H4-H6), siTEBP (A7-A9), siBAD (B7-B9), siBCL-xL (C7-C9), siBCL2 (D7-D9), siCIAP1 (E7-E9), siSmac/Diablo (F7-F9), siTNFR1 (G7-G9), siTRAF2 (H7-H9), siTRADD (A10-A12), siFADD (B10-B12), siRIP2 (C10-C12), siNFKB1A (D10-D12), siNFKB1B (E10-E12), siIKBKE (F10-F12), siIKBKB (G10-G12). White outlines in FIG. 7 represent triplicate wells for selected siRNA-treated samples shown in (B). FIG. 7A shows a close-up view of cells from 1 of 12 images acquired for samples treated with (from left to right) no siRNA, siAKT1, a negative control siRNA (IX), siHSP90beta and siBCL-xL, demonstrating the dynamic range of the effects of knockout of exogenous and endogenous targets on the reporter assay.

### Results

Using the methods described above, the effects of gene silencing on the Akt:Hsp90 protein complexes were investigated. FIG. 7A is a plate view showing the results obtained with 30 separate siRNA pools (Dharmacon, Boulder Colo.) optimized to target specific signal transduction mediators. The results clearly demonstrate that siRNA-mediated silencing of specific signaling molecules has profound and measurable effects on assays, such as those shown here, that reflect signal transduction pathway activity. Increases or decreases in assay activity are evidenced by increased or decreased fluorescence intensity (green channel). Nuclei are stained with Hoechst (blue channel) demonstrating that changes in fluorescence intensity are not a result of cell loss. The assay described in FIG. 7 measures the AKT/HSP90beta complex. Activity in this assay directly reflects the activity of this branch of the AKT pathway. PCA assays are unique in this capacity to simultaneously report on the levels and interaction of two specific members of known and novel protein complexes. As expected, in wells transfected with an siRNA pool targeting a constituent of the PCA, fluorescence signals are decreased to near background levels (siAKT1 and siHSP90β; FIG. 7B). These results indicate that the siRNAs effectively abrogate levels of the messenger RNA and protein against which they were designed, and further demonstrate that mRNA expressed as a PCA fusion is efficiently targeted by siRNA. Expanded images also show that targeted siRNA effects are specific; the untreated and control siRNAXI images show similar levels of fluorescence intensity (FIG. 7B).

In addition to the predicted results of siRNAs directly targeting the assay components, novel, biologically relevant and quantitative results are obtained by combining a signaling assay with an annotation reagent targeting a distinct step in the pathway (i.e., a step not represented in the assay itself). For example, treatment of cells with an siRNA pool targeting the anti-apoptotic protein Bcl-xL (FIG. 7B) led to an increase in activity of the Akt1/HSP90β assay. Connections between Bcl-xL and AKT have been previously implied, but direct signaling links have not been demonstrated [Nimmanapalli et al., Blood 102(1):269-75, 2003]. The direct measurement of the cellular signal transduction pathway effects of annotation reagents such as siRNA is a significant advance over currently employed methods, such as analysis of gene transcription. Here we obtain information about the nature of the signaling networks that defines how protein targets operate in their native environment.

This example shows the results of assessing the effects of different annotation reagents on a single signaling event. This strategy can also be used to assess the effects of one (or multiple) annotation reagents across a broad spectrum of analyses of cellular signal transduction activity.

### Example 3

In a further demonstration of large-scale pathway mapping, we demonstrated the effects in human cells of an siRNA pool that silences the gene encoding the known co-chaperone protein, Cdc37.

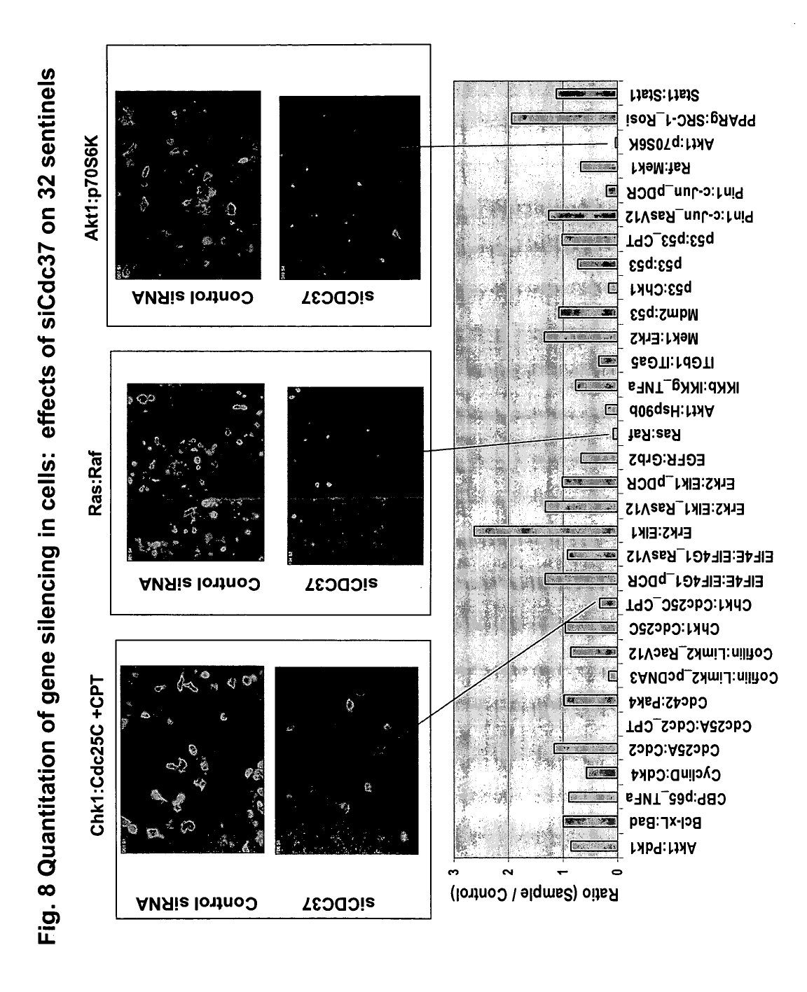

FIG. 8 shows the results of a single annotation reagent, an optimized siRNA pool designed to silence Cdc37, against a panel of 32 assays. These assays report on diverse cellular activities. The effect of the Cdc37 siRNA (40 nM) on the fluorescence intensity of the different protein complexes (assessed by PCA) is plotted relative to the effect of a negative control siRNA. A value of 1 represents no change relative to the control. The 32 assays are represented on the x-axis, and are also listed in Table 2. Silencing of the Cdc37 gene by siRNA significantly decreased the fluorescence intensity of 8 different protein complexes in human cells, many containing proteins that are known to bind to Cdc37 (e.g. Akt1, Raf1, Chk1, etc). Representative images of the effect of siCdc37 on the Chk1:Cdc25C (+CPT), Ras:Raf and Akt:p70S6K assays are shown in FIG. 8 relative to the negative control for each assay.

As discussed above, complexes of HSP90, CDC37 and their client proteins lead to increased activity and stabilization of the client protein. Disruption of the HSP complexes leads to inactivation and degradation of the client protein. Therefore, an assay reporting on HSP client protein level or activity could reflect these changes, allowing novel proteins to be mapped to these pathways. Indeed, FIG. 8 shows three separate examples of client protein modulation by CDC37 ablation. The protein kinases Chk1, Raf-1, and Akt1 are HSP90 client proteins (Workan, P. Curr. Cancer Drug Targets, 3(5):297-300, 2003; Arlander, S. J. et al., J. Biol. Chem. 278(52):52572-7, 2003.) Therefore, complexes with the HSP90 co-chaperone CDC37 are essential for their activity and stability.

Treatment of cells with an siRNA pool targeting CDC37 led to a dramatic decrease in the activity of assays reporting on the level and activity of these proteins. These assays uniquely report on specific aspects of target protein biology. For example, all of these kinases interact with multiple substrate proteins; these assays report individually on specific interactions rather than on total kinase level or activity. The quantitative nature of these assays is also shown and is being used to construct detailed pharmacological profiles, and a complete map, of the pathways in which these proteins participate. The graphical display of results indicates that this approach is a unique, rapid means by which the cellular targets of a particular annotation reagent can be determined.

TABLE 2

| | | | | Reporter 1 | | Reporter 2 |
|---|---|---|---|---|---|---|
| Assay | | | Gene 1 | Fusion | Gene 2 | Fusion |
| # | PCA Pair | Stimulus, conc (time) | Accession | Orientation | Accession | Orientation |
| 1 | Akt1:Hsp90β | | NM_005163 | C | NM_007355 | C |
| 2 | Akt1:p70S6K | | NM_005163 | C | NM_003161 | N |
| 3 | Akt1:Pdk1 | | NM_005163 | N | NM_002613 | C |
| 4 | Bcl-xL:Bad | | NM_138578 | C | NM_004322 | N |
| 5 | IKKβ:IKKγ | | XM_032491 | N | NM_003639 | N |
| 6 | CBP(aa | TNFα, 50 ng/ml (0.5 hr) | S66385 (nt | N | NM_009045 | N |
| | 1 . . . 771):p65 | | 1 . . . 2313) | | | |
| 7 | EGFR:Grb2 | | NM_005228 | C | NM_002086 | C |
| 8 | Stat1:Stat1 | | NM_007315 | C | NM_007315 | N |
| 9 | Ras:Raf | | NM_005343 | N | NM_002880 | C |
| 10 | Raf:Mek1 | | NM_002880 | C | Z16415 | C |
| 11 | Mek1:Erk2 | | Z16415 | C | NM_002745 | C |
| 12 | Erk2:Elk1 | | NM_002745 | C | NM_005229 | C |
| 13 | Erk2:Elk1 | pDCR¹ | NM_002745 | C | NM_005229 | C |
| 14 | Erk2:Elk1 | pDCR.RasV12² | NM_002745 | C | NM_005229 | C |
| 15 | Pin1:c-Jun | pDCR¹ | NM_006221 | C | NM_002228 | C |
| 16 | Pin1:c-Jun | pDCR.RasV12² | NM_006221 | C | NM_002228 | C |
| 17 | EIF4E:EIF4G1 | pDCR¹ | NM_001968 | C | NM_198244 | C |
| 18 | EIF4E:EIF4G1 | pDCR.RasV12² | NM_001968 | C | NM_198244 | C |
| 19 | ITGβ1:ITGαS | | NM_002211 | C | NM_002205 | C |
| 20 | Cdc42:Pak4 | | NM_001791 | N | NM_005884 | C |
| 21 | Cofilin:Limk2 | pcDNA3 | NM_005507 | C | BC_013051 | N |
| 22 | Cofilin:Limk2 | pcDNA3.RacV12³ | NM_005507 | C | BC_013051 | N |
| 23 | Chk1:CDC25C | | NM_001274 | N | NM_001790 | C |
| 24 | Chk1:CDC25C | CPT, 500 nM (18 hr) | NM_001274 | N | NM_001790 | C |
| 25 | Chk1:p53 | | NM_001274 | N | NM_000546 | C |
| 26 | Cdc25A:Cdc2 | | NM_001789 | C | NM_001786 | N |
| 27 | Cdc25A:Cdc2 | CPT, 500 nM (18 hr) | NM_001789 | C | NM_001786 | N |
| 28 | p53:p53 | | NM_000546 | C | NM_000546 | C |
| 29 | p53:p53 | CPT, 500 nM (18 hr) | NM_000546 | C | NM_000546 | C |
| 30 | Mdm2:p53 | | NM_002392 | N | NM_000546 | C |
| 31 | PPARγ:SRC-1 | Rosiglitazone, 15 uM | NM_138712 | C | U40396 (nt | N |
| | | (1.5 hr) | | | 624 . , . 1256) | |
| 32 | CyclinD:CDK4 | | NM_053056 | N | NM_001791 | C |

### Example 4

Annotation of Gene Function Through the Expression of Mutationally Activated Proteins

In addition to annotation technologies and reagents that modulate the expression or stability of RNA, many methods exist for direct modulation of protein activity. These methods ca also be used to modulate protein expression or activity in cells in conjunction with efforts to annotate novel genes and targets. Exogenous expression of wild-type (native) or mutationally-activated proteins has been used extensively to mimic the active state of a particular protein in living cells. This can be achieved by transfecting cells with plasmid-based expression vectors, or infecting with a retrovirus, adenovirus, or lentivirus encoding the protein of interest. We demonstrated this aspect of the invention by measuring the effects of a mutationally activated Ras oncogene on proteins downstream of Ras in human cells. First, protein-fragment complementation assays were constructed for two different pairs of proteins: JNK2/Jun and MEK/ERK. The indicated PCA pairs (JNK2/Jun and MEK/ERK) were co-transfected either with empty vector (PDCR) or with the same vector encoding an activated mutant of H-Ras (RasV12). The mutation of Ras at Glycine 12 in the wild-type sequence to Valine changes the strucutre of the protein in this hydrophobic region, leading to a dramatic decrease in GTP hydrolysis (Futatsugi, N. and Tsuda, M., Biophysical Journal, 81(6):3483-8, 2001). The net result of these changes are Ras proteins that exist constitutively in the GTP-bound state, leading to activation of pathways downstream of Ras proteins such as ERK/MAP kinase pathway activation (Leevers S J and Marshall, C J. EMBO J. 11(2): 569-574, 1992) and JNK/stress-activated kinase pathway activation (Westwick, J. K., Cox, A. D., Der, C. J., Cobb, M. H., Hibi, M., Karin, M., and Brenner, D. A. Proc. Natl. Acad. Sci. USA 91(13): 6030-4, 1994). The interaction of MEK (also known as MAP kinase-kinase) and its substrate ERK (also known as MAP kinase) is a key step in the signal amplification from Ras proteins to the nucleus. In the parallel JNK/stress-activated pathway, Ras signal are amplified through a series of kinases to the ultimate step where JNK activates the transcriptional activity of nuclear proteins such as Jun. Thus, one would expect transfection of the activated Ras mutant (RasV12) to elicit increases in the activity of signaling nodes downstream.

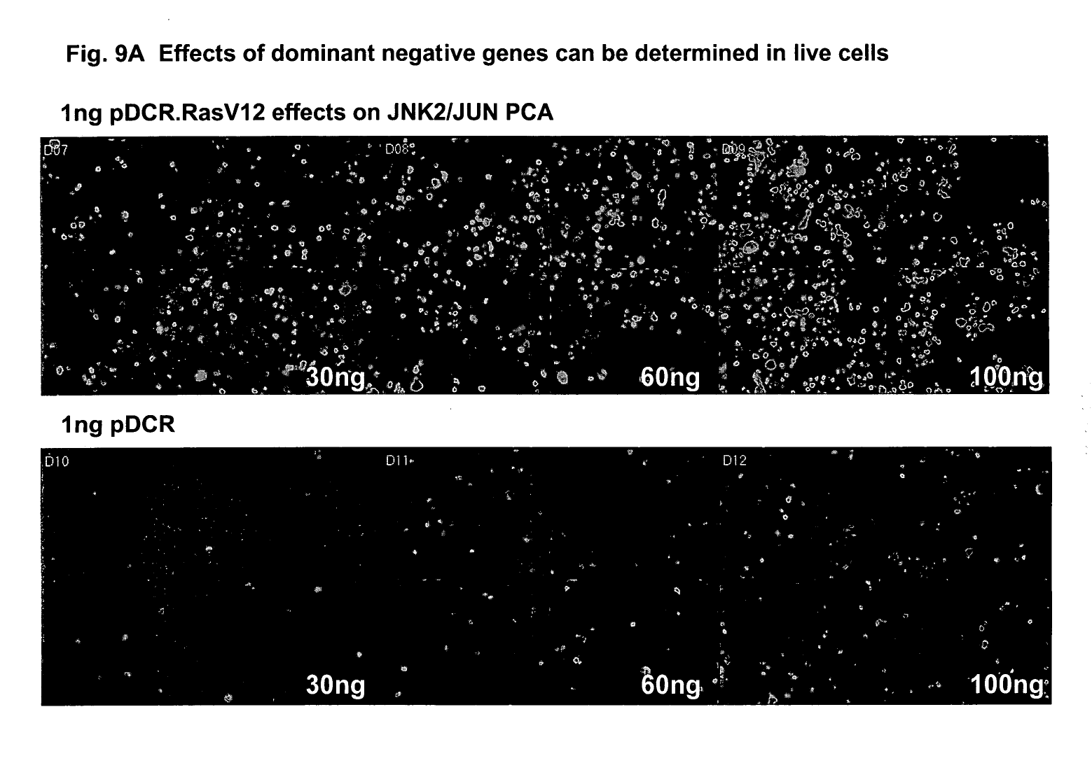

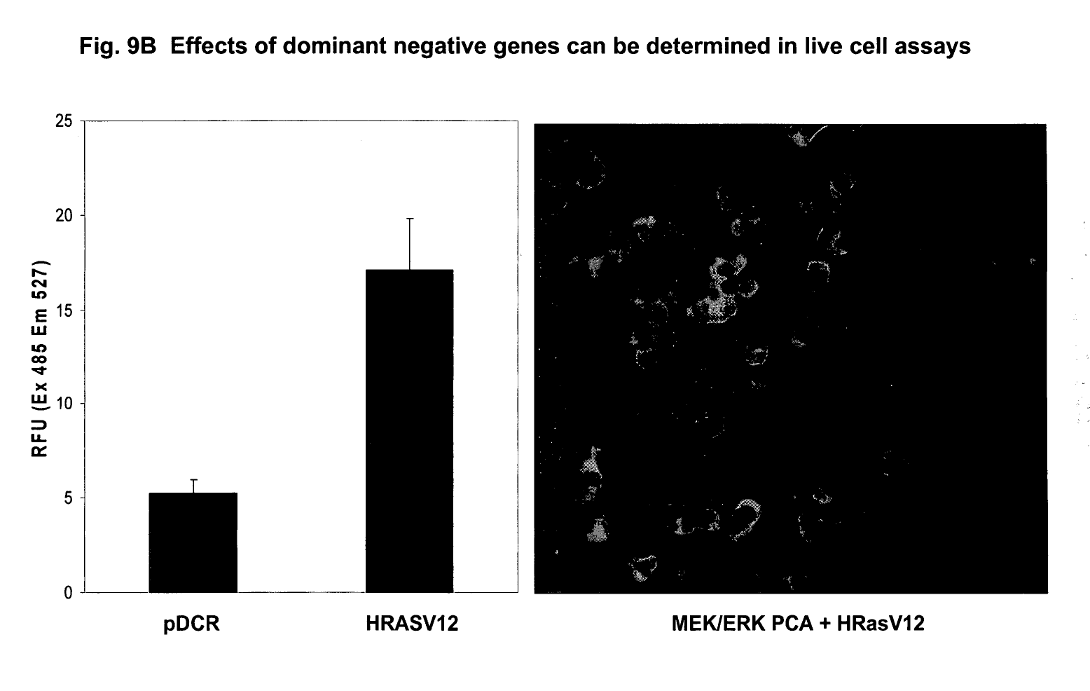

Indeed, as shown in FIG. 9, both the JNK2/Jun (A) and MEK/ERK (B) PCAs are activated in the presence of RasV12. In (A), the experiment was performed with increasing quantities of the PCA pairs (30 ng, 60 ng, or 100 ng of total PCA-pair DNA transfected, with 1 ng of either pDCR (empty vector) or pDCR-Ras12V co-transfected). Transfections were performed using the Lipofectamine 2000 reagents (Invitrogen, Carlsbad Calif.) according to the manufacturers protocol. 48 hours after transfection, cells were stained with Hoechst and subjected to fluorescence imaging with a 20× objective and settings of excitation 480/40 nm, emission 535/50 nm, on a Discovery-1 automated fluorescence microscope (Molecular Devices Corp., Sunnyvale, Calif.). In (B), cells were transfected with MEK and ERK cDNAs (60 ng total DNA; each fused to a fragment of the YFP cDNA, as described in [Yu et al. '04]). The cells were co-transfected with 1 ng of pDCR (empty expression vector; data not shown) or an equivalent amount of pDCR-Ras12V using Lipofectamine 2000, stained and imaged as described above. Quantitation of fluorescence is shown in (B), left panel, and was performed on a Gemini EM plate reader set at Ex: 485 nM, Em:527 nM. In both cases the RasV12-transfected cells demonstrated a striking increase in the level of fluorescence generated by the PCA. In (B), transfection of RasV12 increased the level of specific fluorescence by approximately 3.5-fold (left panel). An image of the RasV12-activated MEK/ERK PCA is shown in the right panel.

In this embodiment of the invention, test reagents that inhibit (e.g., siRNAs, dominant negative proteins, or inhibitory peptides) or activate (e.g. over-expressed wild-type or mutationally activated alleles) signaling proteins or complexes are combined with an assay reporting on the activity of a specific signaling node or process. This combination enables determination of the functional and spatial relationship of the proteins affected by the test reagent and the proteins assessed by the assay. In the example described above, co-transfection of the Ras protein has a measurable effect on assay activity due to the fact that it exists upstream of the proteins in the two assays tested (MEK, ERK, JNK2 and Jun). In addition, these results indicate the functional role of Ras in this process; it is not simply connected to the downstream proteins, but potentiates their interaction and activity. In general, the approach enables determination of hierarchical relationships of proteins within pathways, connections between signaling processes (“cross-talk”), and the functional result (activation, inhibition, translocation, or no change) of the modulation of a particular protein.

### Example 5

### High Content Assays for Use in Conjunction with Annotation Technologies

It will be appreciated by a person skilled in the art that the ability to select from among a variety of live cell assays in conjunction with a gene annotation experiment makes the invention particularly useful for drug discovery on a large scale. However, it is necessary that the assay be capable of measuring the dynamic changes in a protein's activity, or its associations, that occur when a pathway is activated or inhibited. We show that changes in individual proteins in response to an annotation reagent can be measured by one or more of the following methods: methods for measuring the association or dissociation of proteins; methods for measuring the translocations of proteins or protein-protein complexes from one subcellular compartment to another; or the post-translational modifications of proteins. Finally such changes should be quantifiable in intact cells, for example, by measurement of the intensity or the subcellular location of a fluorescent or luminescent signal within the cells. The intensity of a fluorescent signal can be measured with a variety of existing instrumentation, including flow cytometry, fluorimetry, and fluorescence plate readers. The subcellular location of a fluorescent signal can be measured with automated microscopes and automated imaging systems (e.g. U.S. Pat. No. 5,989,835; U.S. Pat. No. 6,172,188).

Tagging of individual proteins with GFP or other intact fluorescent proteins has been widely used to establish high-content assays. Such assays have been used to monitor the redistribution of proteins in response to agonists, antagonists and inhibitors (see van Roessel P & Brand A H, Nature Cell Biol 4: E15-E20. 2002; Tavare J. M., Fletcher L. M., & Welsh G. I., J. Endocrinol. 170: 297-306, 2001; and references therein). In addition such assays have been used in conjunction with siRNA, although primarily to assess the success or extent of silencing of the product of the gene that is silenced. For this purpose the protein that is targeted for silencing is tagged with GFP; the disappearance of fluorescence signal following gene silencing reagent is used as an indication of the success of the gene silencing. Although GFP tagging technology is in common use, the prior art is remarkably silent on the use of such assays to link proteins to other proteins that function upstream or downstream of the targeted protein in a biochemical pathway. In the present invention we demonstrate that redistribution of proteins in complexes downstream of the target of the annotation reagent can be used to map genes into pathways. The process is identical to that depicted in FIG. 6 in that high-throughput or high-content assays proteins of interest are used to construct the pharmacological profiles. Either individual tagged proteins or pairs of tagged proteins can be used in conjunction with a chosen annotation reagent, so long as the proteins respond dynamically to inhibition or activation of the pathway of interest. Fluorescence intensity or subcellular redistribution can be used to construct such pharmacological profiles and to map novel genes into biochemical pathway mapping. In the case of redistribution of signal in response to a pathway probe, the quantitative parameter used to construct the pharmacological profile is the ratio of the fluorescence signal in one compartment vs. another compartment.

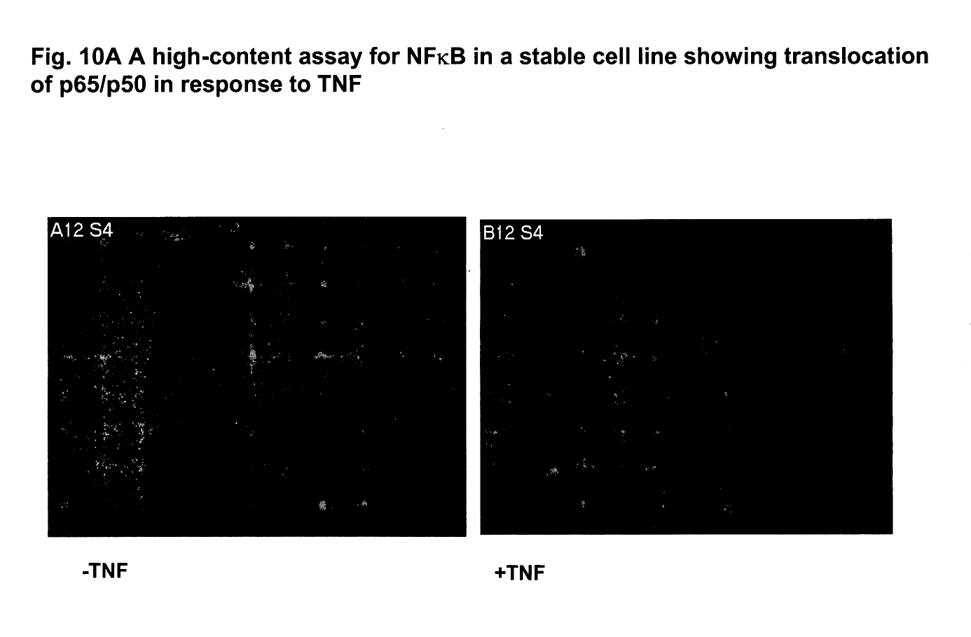

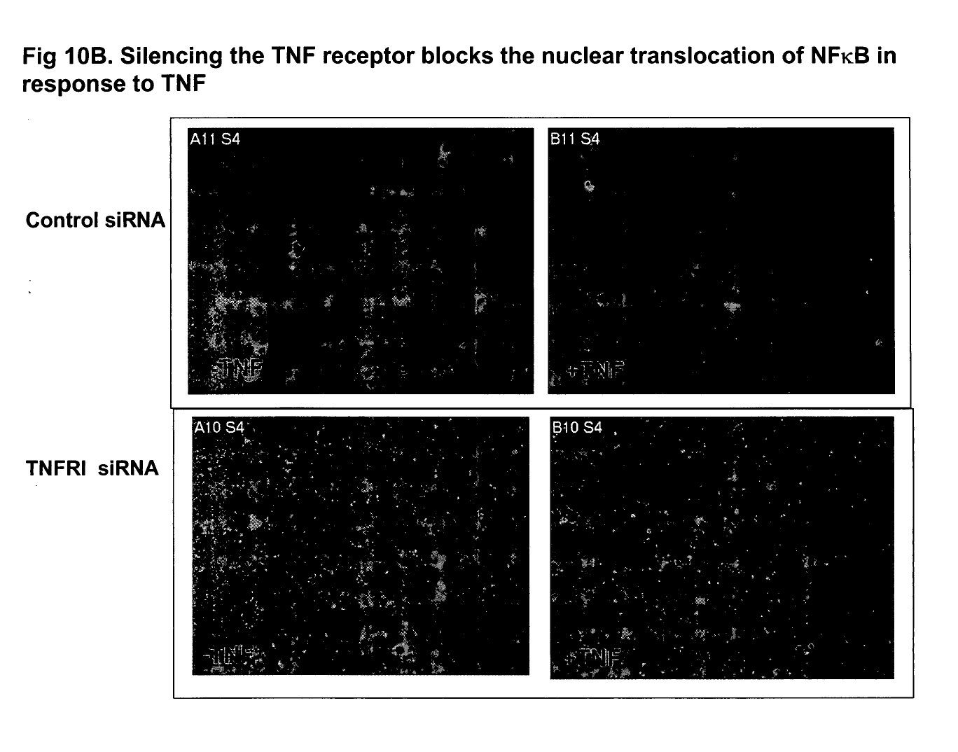

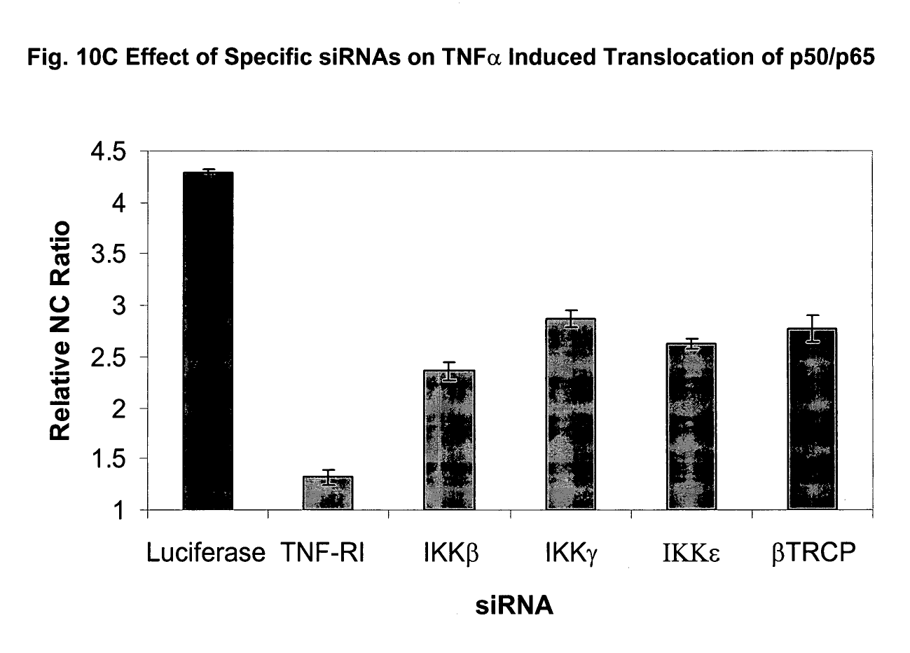

For example, in the case of the NFkappaB transcription factor, redistribution of the p65 subunit from the cytosol to the nucleus occurs in response to tumor necrosis factor (TNF) (Ding, J. G. F., et al., J. Biol. Chem. 273: 28897-28905, 1998). We show that this event is blocked by silencing of genes ‘upstream’ of NFkappaB in the TNF pathway, such as the TNF receptor; demonstrating that the NFkappaB complex is functionally linked to the TNF receptor. These effects, and the corresponding pharmacological profiles for these proteins, can also be determined by measuring the amount of p65 in the nucleus vs. the amount of p65 in the cytosol in the absence and presence of an annotation reagent. Such measurements can be made, for example, by expressing the p65 subunit of NFkappaB as a fusion with a luminophore—such as GFP or YFP—and monitoring the redistribution of signal generated by the p65 fusion protein; or alternatively co-expressing p65 tagged with a fragment of GFP or YFP along with the other (p50) subunit of NFkappa B tagged with the complementary fragment of GFP or YFP, and performing a protein-protein interaction assay as we show in FIG. 10. Either of these high-content assay methods allow the activity of p65 to be linked to the TNF pathway in intact cells. Commercial assays and kits for measuring the redistribution of GFP-tagged proteins are distributed by Cellomics, Inc. and by BioImage (Denmark). Any of these or similar assays are suitable for use in conjunction with the present invention.

FIG. 10 shows the effect of silencing a receptor on proteins in the signaling pathway linked to the receptor. We used HEK293 cells, which are responsive to TNF, and constructed a stable cell line using a YFP PCA for p65/p50 (Yu et al., 2004). In the absence of TNF, the p65/p50 complex is predominantly located in the cytoplasm of the cells (FIG. 10A, left panel). Treatment of the cells with TNF leads to a redistribution of the p65/p50 complex into the nucleus (FIG. 10A, right panel). The effect of TNF can be abrogated by 48 hours' pretreatment with an siRNA targeting the TNF receptor (TNFR1 siRNA; FIG. 10B right). In contrast, a control (siLuc) siRNA does not abrogate the TNF response. These results demonstrate that p65 and p50 are functionally linked to the TNF receptor. FIG. 10C shows that a variety of other siRNA's targeting genes encoding proteins that are known to act ‘upstream’ of p65 and p50 in the TNF pathway also knock down the activity of the pathway, as assessed by this high-content assay.

Similar approaches can be applied to identify novel components of the TNF pathway using the approaches shown in FIG. 6. First, a novel protein that interacted with p65, p50, the TNF receptor protein, or other known elements of the TNF pathway, could be identified by performing a protein-protein interaction assay using one or more of the methods described herein. Second, the functional relationship between the novel protein and the TNF response could be validated by performing the assay for the novel protein complex in the absence or presence of TNF and one or more gene annotation reagents, and using the results to construct a pharmacological profile for the novel protein such as was done for p65/p50 in FIG. 10C. The pharmacological profile for the novel protein is then compared to the pharmacological profile for p65, p50, or any other protein that is known to be involved in the TNF pathway. If the pharmacological profile for the novel protein is similar to the pharmacological profile for the established protein, the novel protein can be inferred to participate in the same pathway.

### Example 6

Using Gene Annotation Technologies in Conjunction with High-Content Assays Based on Immunofluorescence

An alternative approach to elucidate the effect of an annotation reagent on a specific signaling process is through the use of immunological reagents. By applying antibodies to fixed cells, one can measure the absolute levels of a particular protein or class of proteins, as well as specific post-translational modifications (e.g. phosphorylation, acetylation, ubiquitination) of a protein or class of proteins. In addition, the sub-cellular localization of a protein or modified protein (or class or proteins) can be assessed in this manner, enabling automated, high-content analyses in a manner similar to that shown for PCA.

To demonstrate the general strategy and its application we studied the ERK mitogen-activated protein kinases, which are key relay points in the transmission of growth factor-generated signals. The canonical growth factor receptor-stimulated pathway is initiated by a cell surface receptor, such as the epidermal growth factor (EGF) receptor tyrosine kinase. Activated receptors bind to adaptor proteins and guanine nucleotide exchange factors, such as the protein SOS. SOS, in turn, activates small GTPases such as Ras, which then lead to phosphorylation and activation of a cascade of kinases including B-Raf and ERKs. By measuring the activity of a distal step in the pathway, such as phosphorylation of ERKs, the activity of upstream steps can be inferred using a strategy identical to that described for FIG. 6.

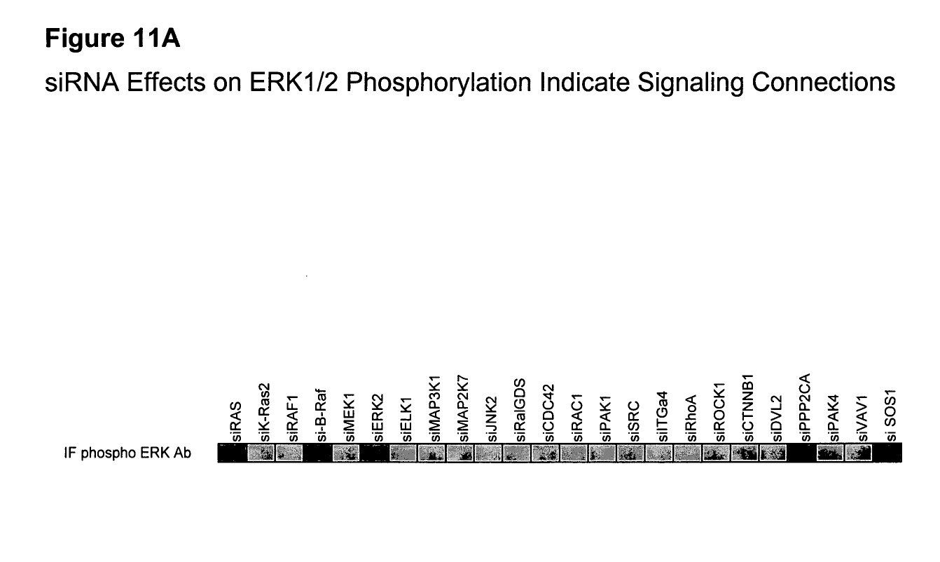

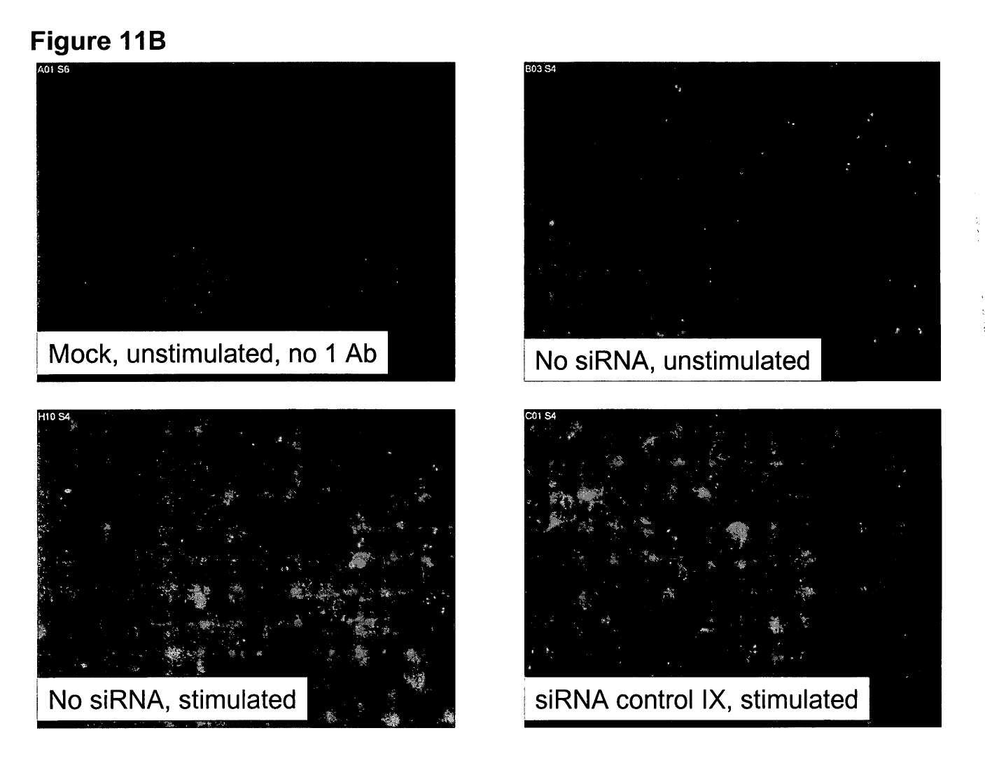

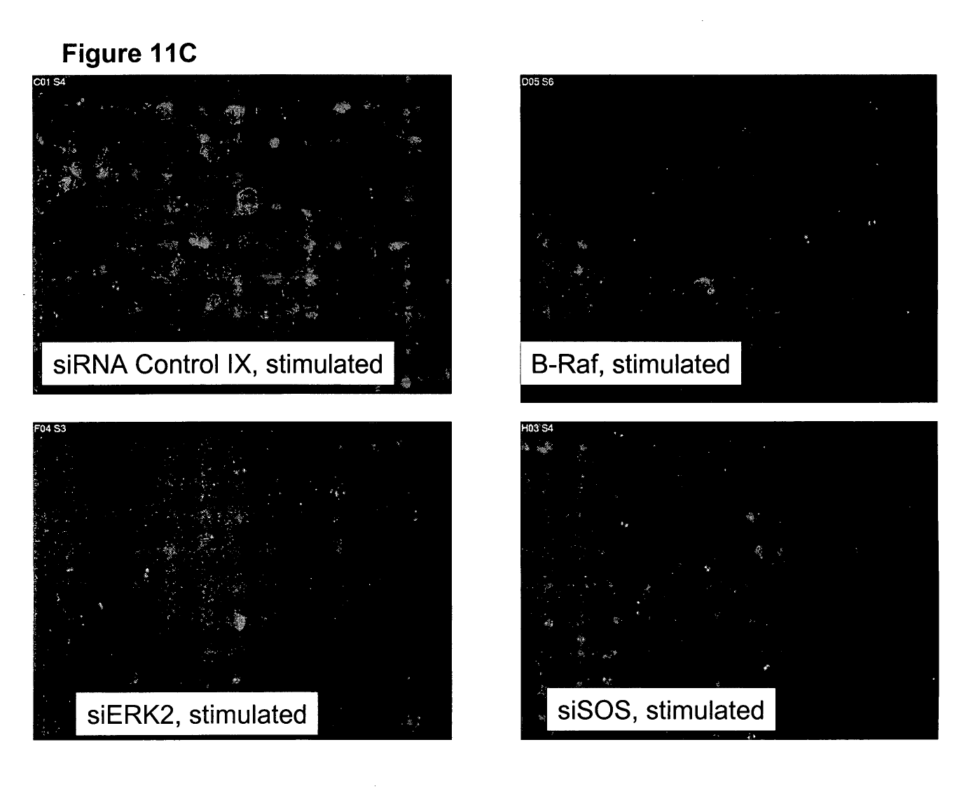

Cells are transfected with a collection of siRNAs, and cultured for 48 hours before fixing. Fixed cells are probed with antibodies that specifically recognize the MAPK/ERK protein kinases only when they are phosphorylated on Threonine 202 and Tyrosine 204 in the activation loop. Phosphorylation of these amino acids has been shown to be necessary and sufficient for kinase activation, and therefore is a surrogate marker for activation of the kinases [Robbins et al.]. Changes in the level and sub-cellular localization of phosphorylated proteins following treatment with an annotation reagent (here siRNA) indicates a functional connection between the annotation reagent and the target of interest. FIG. 11 shows an example of this approach. Here, 24 individual siRNA pools were transfected into human HEK cells, and their effects on the phosphorylation of the ERK1 and ERK2 protein kinases was assessed by immunofluorescence analysis.

### Methods

Human HEK 293T cells were plated at 12,000 cells/well, and transfected with 40 ng of siRNA pools targeting the indicated proteins using Lipofectamine 2000 (Invitrogen, Carlsbad Calif.). 24 hours after transfection, cells were cultured in 0.25% serum for 24 hours, then treated with EGF (Sigma; 100 ng/ml) for 5 minutes prior to fixation. For immunofluorescence, cells were rinsed with PBS, fixed with 4% formaldehyde in PBS, and permeabilized using 0.25% Triton X-100. Non-specific immunoreactivity was blocked by incubating cells with 3% BSA in PBS. To detect phosphorylated target proteins, cells were incubated with phospho-specific primary antibodies for 2 hrs. Rabbit anti phospho-p42/44 Map kinase (T202/Tyr204) was obtained from Cell Signaling Technologies, Beverly Mass. (Cat. #9101). These antibodies were used at a dilution of 1:50 in blocking buffer. Cells were subsequently rinsed with PBS/BSA, followed by incubation with Alexa-488 conjugated goat anti-rabbit antibody. Cells were counterstained with nuclear marker Hoechst 33342 (Molecular Probes). All liquid handling was performed using BioMEK FX (Beckman). Automated fluorescence image acquisition was carried out using Discovery-1 (Universal Imaging/Molecular Devices).

### Results

Gene silencing, by treatment with several of the indicated siRNAs, resulted in significant inhibition of ERK phosphorylation as assessed by immunofluorescence. These included siSOS, siRas, siB-Raf, siERK, and siPPP2CA (an siRNA targeting the catalytic subunit of protein phosphatase 2A). Interestingly, the proteins targeted by all of the siRNAs have been previously shown to regulate this pathway. The application of this method is shown in FIGS. 11B and 11C. The level of phospo-ERK in unstimulated cells (relative to cells probed with the secondary antibody only) is shown in FIG. 11B, right top panel. EGF treatment results in a large increase in phosphorylated ERK, as expected (FIG. 11B, bottom left panel). Treatment of cells with a control non-specific siRNA 48 hours before stimulation does not change the level of phosphorylated ERK (FIG. 11B, bottom right panel). However, treatment with siRNA targeting B-Raf or SOS, two obligate upstream mediators of the ERK signaling cascade, significantly attenuate ERK phosphorylation. These results demonstrate the utility of the approach. Any siRNA causing a similar effect would either be an intermediate constituent on the pathway between growth factor stimulation and the protein(s) assessed, or would be the protein itself. In addition to simple measurements of protein level or post-translational modification, this approach is amenable to assessment of changes in sub-cellular localization elicited by an annotation reagent. For example, in FIG. 11C both siB-Raf and siSOS cause a decrease in the phosphor-ERK signal. More detailed inspection of these images reveals that the siB-Raf causes a general decrease in phospho-ERK across the cells, whereas siSOS-mediated decrease in phosphor-ERK is particularly pronounced in the cytoplasm of cells. It is well-known that ERK targets specific substrate proteins that reside in particular compartments of the cell; therefore these relative changes in sub-cellular location of phosphorylated ERK are functionally important.

By combining annotation reagents with a specific signal transduction readout, such as ERK phosphorylation, connections between the annotation reagent and the assayed protein are derived. Finally, as these assays rely on functional activity (in this case, phosphorylation of a protein) the hierarchy of the pathway is revealed. Thus, the approach demonstrates a physical connection, but also functional connections between the annotation reagent and the assayed protein(s).

In addition, the hierarchical structure of signaling pathways are becoming well-known, thus this approach generates testable hypotheses regarding not just the analytes tested, but proteins known to be functionally linked to the analyte. In the example described here, we conclude that if a specific siRNA has an effect on the phosphorylation of ERKs, the gene it targets is also functionally linked to ERK-regulated gene expression. In addition, the targeted gene may also be linked to proteins leading to ERK activation, such as the MAP kinase kinase (MEK), Raf kinases, and Ras oncoproteins. Using the tools described here, we also test these hypotheses in order to determine the site of action of specific genes. Thus, by combining the modulation of various targets by siRNA-mediated gene product ablation (or with other annotation reagents) and pathway-based analysis of protein levels and activity, we can directly ascribe function to any known protein.

This aspect of the invention has the advantage of allowing the identification of the post-translational modification status of either endogenous or exogenous proteins. The commercial availability of a variety of highly specific antibodies for individual proteins enables this aspect of the invention, in conjunction with automated methods, in a manner nearly identical to that accomplished with PCA. Immunofluorescence methods can be used in conjunction with this aspect of the invention. We show that IF can be applied to intact cells following gene or protein silencing, inactivation, or activation. IF can be used in conjunction with antibodies that bind differentially to their targets depending on the post-translational modification status of the target, as shown here, or that bind to epitope-tagged proteins such as HA-tagged proteins. Antibodies that are useful for the present invention can be generated using standard monoclonal antibody methods; additionally, many useful antibodies are already available from commercial suppliers including Cell Signaling Technology Inc., Santa Cruz Biotechnology Inc., Roche Molecular Biochemicals, Inc., and BioSource Inc.

Alternative approaches to the detection of post-translational modifications include methods capable of measuring the attachment of the modifier to the protein of interest. For example, protein-fragment complementation assays and similar interaction assays can be used to measure ubiquitination of proteins. In this case the ubiquitin polypeptide is tagged with one complementary fragment of a reporter and the protein of interest is tagged with the other fragment of the reporter. Ubiquitination of the protein of interest then generates a fluorescent (or luminescent) signal that can be quantified and localized in intact cells. Any sensitive fluorescent or luminescent method capable of detecting post-translational modifications of individual proteins as a result of signaling processes can be used in conjunction with the present invention. A number of such methods have been described in the literature (Blake R A., Curr Opin Pharmacol 1(5): 533-539, 2001; Gritzapis A D et al., Breast Cancer Res Treat. 80(1): 1-13, 2003; Gratama J W et al., Cytometry 33(2): 166-178, 1998).

### Additional Methods for Use in Conjunction with the Invention

The most widespread fluorescent, cell-based protein-protein interaction assay is based on the phenomenon of fluorescence resonance energy transfer (FRET) or bioluminescence resonance energy transfer (BRET) (for a review see Miyawaki A. & Tsien R. Y. in: Methods in Enzymology 327: 427-500, 2000). In a FRET assay the genes for two different fluorescent reporters, capable of undergoing FRET are separately fused to genes encoding of interest, and the fusion proteins are co-expressed in live cells. When a protein complex forms between the proteins of interest, the fluorophores are brought into proximity if the two proteins possess overlapping emission and excitation, emission of photons by a first, “donor” fluorophore, results in the efficient absorption of the emitted photons by the second, “acceptor” fluorophore. The FRET pair fluoresces with a unique combination of excitation and emission wavelengths that can be distinguished from those of either fluorophore alone in living cells. As specific examples, a variety of GFP mutants have been used in FRET assays, including cyan, citrine, enhanced green and enhanced blue fluorescent proteins. With BRET, a luminescent protein, for example the enzyme Renilla luciferase (RLuc) is used as a donor and a green fluorescent protein (GFP) is used as an acceptor molecule. Upon addition of a compound that serves as the substrate for Rluc, the FRET signal is measured by comparing the amount of blue light emitted by Rluc to the amount of green light emitted by GFP. The ratio of green to blue increases as the two proteins are brought into proximity. Quantifying FRET or BRET-can be technically challenging and use in imaging protein-protein interactions is very limited due to the very weak FRET signal. FRET often does not produce a very bright signal because the acceptor fluorophore is excited only indirectly, through excitation of the donor. The fluorescence wavelengths of the donor and acceptor must be quite close for FRET to work, because FRET requires overlap of the donor emission and acceptor excitation. Newer methods are in development to enable deconvolution of FRET from bleedthrough and from autofluorescence. In addition, fluorescence lifetime imaging microscopy (FLIM) eliminates many of the artifacts associates with quantifying simple FRET intensity.

A variety of high-throughput assays have been constructed based either on activity of wild-type beta-galactosidase or on the phenomenon of alpha- or omega-complementation. Beta-gal is a multimeric enzyme which forms tetramers and octomeric complexes of up to 1 million Daltons. beta-gal subunits undergo self-oligomerization which leads to activity. This naturally-occurring phenomenon has been used to develop a variety of in vitro, homogeneous assays that are the subject of over 30 patents. Alpha- or omega-complementation of beta-gal, which was first reported in 1965, has been utilized to develop assays for the detection of antibody-antigen, drug-protein, protein-protein, and other bio-molecular interactions. Until recently, the adaptation of beta-gal complementation to live cell assays has been limited because the phenomenon occurs naturally, resulting in significant background activity. The background activity problem has been overcome in part by the development of low-affinity, mutant subunits with a diminished or negligible ability to complement naturally, enabling various assays including for example the detection of ligand-dependent activation of the EGF receptor in live cells.

PCA represents a particularly useful assay strategy for high-throughput or high-content assays. PCA enables measurements of the association, dissociation or sub-cellular localization of protein-protein complexes within the cell. PCA enables visualization and quantification of the amount and subcellular location of protein-protein complexes in living cells. With PCA, proteins are expressed as fusions to engineered polypeptide fragments, where the polypeptide fragments themselves (a) are not fluorescent or luminescent moieties; (b) are not naturally-occurring; and (c) are generated by fragmentation of a reporter.

Michnick et al. (U.S. Pat. No. 6,270,964) taught that any reporter protein of interest can be used in PCA, including any of the reporters described above. Thus, reporters suitable for PCA include, but are not limited to, any of a number of enzymes and fluorescent, luminescent, or phosphorescent proteins. Small monomeric proteins are preferred for PCA, including monomeric enzymes and monomeric fluorescent proteins, resulting in small (˜150 amino acid) fragments. Since any reporter protein can be fragmented using the principles established by Michnick et al., assays can be tailored to the particular demands of the cell type, target, signaling process, and instrumentation of choice. Finally, the ability to choose among a wide range of reporter fragments enables the construction of fluorescent, luminescent, phosphorescent, or otherwise detectable signals; and the choice of high-content or high-throughput assay formats. For any reporter of interest various useful PCA fragments can be created using the methods taught in U.S. Pat. No. 6,270,964 (Michnick et al.), and the fragments can be further engineered to generate a brighter signal upon fragment reassembly. In the present application, protein fragments were generated either by PCR or were generated synthetically (by oligonucleotide synthesis) to create fragments with the desired assay properties. PCA fragments that reconstitute enzymes can be used in conjunction with various substrates or probes to generate assays with different spectral properties. Other reporters suitable for PCA are described in Table 1 and in Michnick et al. (U.S. Pat. No. 6,270,964) and include monomeric enzymes and fluorescent, luminescent or phosphorescent proteins. Also, PCAs based on fragments of antigens or antibodies can be created and used in conjunction with simple detection schemes. For example, PCAs based on fragments of a non-native antigen could be constructed such that a protein-protein interaction results in reconstitution of an epitope that can be detected with an antigen conjugated to a detectable moiety such as biotin or fluorescein. Similarly, PCAs based on fragments of an antibody could be constructed such that a molecular interaction results in reconstitution of a functional antibody that binds to an antigen conjugated to a detectable moiety such a fluorophore. Any of these and similar reporters can be used, and modifications thereof, in conjunction with the present invention.

Finally, the high-content or high-throughput assays described herein can be used in conjunction with a variety of existing, automated systems for drug discovery, including existing high-content instrumentation and software such as that described in U.S. Pat. No. 5,989,835. For expression of proteins, a variety of vector systems (plasmid, retroviral and adenoviral and systems) can be used that are well known in the art. Transfection of cells can be accomplished by any of a variety of methods including chemical transfection methods (Fugene, Lipofectamine and other reagents) and electroporation. Protein reagents can be used if they can be delivered into cells; microinjection methods can be used, and proteins can be tagged with peptides that enable cell permeability. All such methods will be well known to those skilled in the art of biochemistry and cell biology.

We have demonstrated the first broadly useful approach to the uses of gene silencing and other gene annotation technologies in living cells. The strategy presented here allows the mapping of disease pathways and functional annotation of novel genes and proteins on a genome-wide scale.

Previous approaches to gene annotation and pathway mapping have utilized yeast two-hybrid analysis or gene expression analysis to derive connections between targets and cellular processes. The approach described herein has many significant advantages. First, two-hybrid analyses have revealed physical connections between proteins as they occur in yeast. However, the effect of pathway perturbations (such as RNAi-mediated target ablation) are unlikely to be observed in the yeast system, as yeast two-hybrid interactions register only if they occur in the nucleus of the cell, not in the context of their normal signaling role. Second, gene expression analyses have been performed in conjunction with protein activation or ablation, including RNAi [Jackson et al.]. However, these analyses report on changes in gene expression that are secondary or tertiary events following modulation of a particular protein target. Deconvolution of the relationship between changes in gene expression, and a specific upstream event leading to that change, are difficult and often impossible. In addition, changes in gene expression correlate poorly with changes in the levels and activity of the proteins they encode. In contrast, the approach described here provides direct evidence of the physical and functional relationships between proteins in various signaling pathways.

The methods exemplified herein are robust; quantitative; and readily automatable. The approach can be used in conjunction with any type of nucleic acid, protein, or chemical reagent or probe in combination with an intact cell assay. Moreover, any cell type or model system suitable for the research of interest can be used for these purposes. For example, PCAs—and immunofluorescence assays—have been performed in bacteria, yeast, fungal, mouse, human and higher plants. Primary and cultured or propagated cells of any tissue of origin can be used. As long as the cells are transfectable, exogenous genes can be expressed using suitable vectors. Therefore the signaling events of interest can be studied in cells that contain the signaling machinery of interest, regardless of the cell type or cell source. This allows cancer pathways to be studied in cancer cells (prostate, breast, colorectal, lung, myelocyte, etc. etc.); insulin signaling to be studied in insulin-responsive cells; immune regulation pathways to be studied in T- and B-cells; cholinergic pathways to be studied in neural cells; and so forth.

The cell-based assays can incorporate any one of a number of measurements of protein activity, so long as the measurement occurs in an intact cell that faithfully recapitulates the pathway of interest. The reagents can be added automatically, for example by automated pipetting systems, providing a high degree of reliability.

The assay can incorporate the measurement the activity, subcellular location, quantity or post-translational modification status of proteins and protein-protein complexes as shown in several examples above. Additionally the assay could involve the measurement of the interaction or formation of a complex between a protein and another molecule such as DNA or RNA. Such assays are possible with PCA or with alternative biomolecular interaction technologies.

The invention allows novel genes and proteins to be mapped into biochemical pathways in living cells and allows disease pathways to be mapped—and novel pharmaceutical targets to be validated—on a large scale. Novel targets can be physically mapped into well-characterized disease pathways, by linking the effect of the gene annotation reagent to the behavior or activity of the novel target. In addition, novel disease pathways can be identified, by linking the effect of any suitable annotation reagent on a cellular phenotype with the effect of the same reagent on novel genes or proteins.

The use of the invention is not limited to the assay format. Multiwell formats, slide formats, microfluidic instrumentation devices and systems, and nanotechnology approaches can be applied in conjunction with the invention. Moreover, any instrumentation suitable for the measurement of the signal generated by the assay can be utilized. Reverse transcription methods can also be used for expression of proteins in conjunction with this invention (e.g. Sabatini, D. M., Erdjument-Bromage, H., Lui, M., Tempst, P. & Snyder, S. H. (1994) Cell 78, 35-43).

The methods shown herein, including PCA, can also be used to construct assays for novel small-molecule inhibitors or activators of these proteins, in order to identify novel anticancer and immunosuppressive drugs from small-molecule, natural product or other compound libraries. The efficacy of a small molecule on a pathway of interest can be assessed by directly comparing the effect of the small molecule to the effect of gene silencing, using a protein-protein interaction assay or any of the other high-content or high-throughput assays specified herein as the readout.

Although the present invention has been described with reference to specific details of certain embodiments thereof, it is not intended that such detail should be regarded as limitations upon the scope of the invention, except as and to the extent that they are included in the accompanying claims.

### REFERENCE LIST

The entire contents including the references cited therein of the following patents and publications are incorporated by reference in their entirety for all purposes to the same extent as if each individual patent, patent application or publication were so individually denoted.

U.S. Pat. No. 6,270,964 Michnick, et al.

U.S. Pat. No. 6,294,330 Michnick, et al.

U.S. Pat. No. 6,428,951 Michnick, et al.

U.S. Pat. No. 5,989,835 Dunlay, et al.

U.S. Pat. No. 6,518,021 Thastrup, et al.

### Publications

Pelletier, J. N., Remy, I. and Michnick, S. W. (1998). Protein-Fragment Complementation Assays: a General Strategy for the in vivo Detection of Protein-Protein Interactions. Journal of Biomolecular Techniques, 10: 32-39.

Remy, I., Pelletier, J. N., Galarneau, A. and Michnick, S. W. (2002). Protein Interactions and Library Screening with Protein Fragment Complementation Strategies. Protein-protein interactions: A molecular cloning manual. E. A. Golemis, editor. Cold Spring Harbor Laboratory Press. Chapter 25, 449-475.

Remy, I., Wilson, I. A. and Michnick, S. W. (1999). Erythropoietin receptor activation by a ligand-induced conformation change. Science, 283: 990-993.

Galarneau, A., Primeau, M., Trudeau, L.-E. and Michnick, S. W. (2002). A Protein fragment Complementation Assay based on TEM1 β-lactamase for detection of protein-protein interactions. Nat Biotechnol, 20: 619-622.

Michnick, S. W., Remy, I., C.-Valois, F. X., Vallee-Belisle, A., Galarneau, A. and Pelletier, J. N. (2000) Detection of Protein-Protein Interactions by Protein Fragment Complementation Strategies, Parts A and B (John N. Abelson, Scott D Emr and Jeremy Thorner, editors) A Volume of Methods in Enzymology. 328, 208-230.

Remy, I. and Michnick, S. W. (2001). Visualization of Biochemical Networks in Living Cells. Proc Natl Acad Sci USA, 98: 7678-7683.

Yu H., West, Keon, Bilter, Owens, Lamerdin, & Westwick (2004) Measuring Drug Action in the cellular context using Protein Fragment Complementation Assays. In: Assays and Drug Development Technologies 1 (6): 811-822.

Schmid, J. A., et al. (2000) Dynamics of NFkappaB and IkappaBalpha studied with green fluorescent protein (GFP) fusion proteins. J. Biol. Chem. 275 (22): 17035-17042.

What is claimed is:

1. A method of annotating the function of a gene or a protein, said method comprising:
(a) contacting a first population of cells with a test reagent, wherein said test reagent is targeted to a particular gene or protein;
(b) measuring the quantity, sub-cellular location, activity, and/or post-translational modification status of a protein or a protein-protein complex of interest in the first population of cells;
(c) measuring the quantity, sub-cellular location, activity and/or post-translational modification status of said protein or protein-protein complex in a second population of cells which has been contacted either with a control reagent or with no reagent;
(d) comparing the results obtained from the first population of cells to the results obtained from the second population of cells;
(e) using the results of step (d) to identify changes in said protein or protein-protein complex caused by said test reagent; and
(f) identifying a protein or proteins as being functionally linked to the gene or protein targeted by said test reagent, if said test reagent causes a measurable change in said protein or protein(s).

2. A method of measuring the effects of a test reagent in a living cell, said method comprising:
(a) contacting a first population of cells with a test reagent, wherein said test reagent is targeted to a particular gene or protein;
(b) measuring the quantity, sub-cellular location, activity, and/or post-translational modification status of a protein or a protein-protein complex in said first population of cells;
(c) measuring the quantity, sub-cellular location, activity and/or post-translational modification status of said protein or protein-protein complex in a second population of cells which has been contacted either with a control reagent or with no reagent;
(d) comparing the results obtained from said first population of cells to the results obtained from said second population of cells;
(e) using the results of step (d) to identify changes in said protein or protein-protein complex caused by said test reagent; and
(f) identifying said test reagent as affecting said protein(s) if said test reagent causes a measurable change in said protein(s).

3. A method of identifying the site of action of a protein within a biochemical pathway, comprising:
(a) constructing a high-content or a high-throughput assay for a first protein of interest;
(b) constructing a high-content or a high-throughput assay for one or more second proteins within a biochemical pathway or pathways;
(c) performing the assays from steps (a) and (b) in the absence and presence of one or more test reagents;
(d) using the results of (c) to establish quantitative pharmacological profiles for each of said protein(s);
(e) comparing the pharmacological profile for said first protein to the pharmacological profiles for each of said second proteins(s);
(f) using the result of (e) to identify the biochemical pathways(s) in which the first protein participates.

4. A method of identifying or validating novel disease pathways and/or therapeutic targets, said method comprising:
(a) contacting a cell, tissue, or organism with one or more test reagents;
(b) determining the biochemical, biological, phenotypic, and/or physiological effects of each of said test reagents;
(c) based on the results of (b), identifying a test reagent with desired effects;
(d) using a method according to any of claims 1-3, identifying the biochemical pathway(s) in which the target of said reagent participates.

5. A method for constructing an assay, said method comprising:
(a) selecting genes encoding proteins that interact;
(b) selecting appropriate reporters or reporter fragments;
(c) fusing or attaching said reporters or reporter fragments separately to the genes encoding said interacting proteins;
(d) expressing said proteins in living cells;
(e) associating said reporters or reporter fragments through interactions of said proteins that are fused or attached to said reporters or said fragments; and
(f) measuring the activity of said reporters in the absence or presence of a test reagent.

6. A method according to claim 5 whereby the reporter is selected from the group consisting of a fluorescent protein, a luminescent protein, a phosphorescent protein, an antigen, an antibody, or an enzyme.

7. A method according to any of claims 1, 2, 3, 4, or 5 wherein said test reagents are selected from the group consisting of a gene silencing reagent, a gene expression reagent, a ribonucleotide, a deoxyribonucleotide, a peptide, a ligand, a polypeptide, a protein, a virus, a chemical, or an antibody.

8. A method for identifying or validating novel pharmaceutical targets comprising:
(A) using a protein-protein interaction assay to identify a first protein that interacts with other proteins within a biochemical pathway of interest;
(B) determining whether said protein actively participates in said pathway, by establishing a pharmacological profile for said interaction and comparing said pharmacological profile with the pharmacological profiles of other interactions in the same pathway.

9. A composition comprising an assay panel, said panel comprising high-content or high-throughput assays for two or more expressed proteins or endogenous proteins in intact cells.

10. An assay panel, said panel comprising two or more fluorescence or luminescence assays for the detection of gene or protein silencing, gene or protein inactivation, or gene or protein activation in an intact cell.

11. A method or an assay according to any of claims 1, 2, 3, 4, 5, 6, 8, 9 or 10 whereby the assay is performed to annotate the function of a novel gene, to map a gene or a protein into a biochemical pathway, to identify disease pathways, or to identify or validate potential pharmaceutical targets.

12. An method or an assay according to any of claims 1, 2, 3, 4, 5, 6, 8, 9 or 10 wherein a fluorescence, bioluminescence, chemiluminescence, phosphorescence or other optically detectable signal is generated in live cells.

13. A method or an assay according to any of claims 1, 2, 3, 4, 5, 6, 8, 9 or 10 wherein microscopy, confocal imaging, flow cytometry, fluorimetry, luminometry, spectroscopy, and/or fluorescence lifetime imaging is used to acquire the signal.

14. An assay according to any one of claims 1, 2, 3, 4, 5, 6, 8, 9 or 10 whereby the assay is carried out in a format selected from the group consisting of a multiwell plate format, a slide formats, a chip formats, a microfluidic format, a nanotechnology format.
